(19) United States
(12) Patent Application Publication
(10) Pub. No.: US 2025/0382357 A1
(43) Pub. Date: Dec. 18, 2025

(54) COMPLEMENT C3 ANTIGEN BINDING PROTEINS

(71) Applicant: Boehringer Ingelheim International GmbH, Ingelheim am Rhein (DE)

(72) Inventors: Leonardo BORRAS, Birmensdorf (CH); Dominik ESCHER, Huenenberg (CH); Stephanie JUNGMICHEL, Oerlikon - Zuerich (CH); Christian LEISNER, Thalwil (CH); Philipp Robert RICHLE, Zuerich (CH); Fabian SCHEIFELE, Maegenwil - Aargau (CH)

(21) Appl. No.: 19/169,344

(22) Filed: Apr. 3, 2025

Related U.S. Application Data
(63) Continuation of application No. 17/669,398, filed on Feb. 11, 2022, now abandoned.

(30) Foreign Application Priority Data
Feb. 12, 2021 (WO) PCT/EP2021/053526

(51) Int. Cl. C07K 16/18

(52) U.S. Cl. CPC C07K 16/18; C07K 2317/565; C07K 2317/569; C07K 2317/76; C07K 2317/92

(57) ABSTRACT

Antigen-binding proteins with specificity to complement C3 and C3b are provided. Methods of treating complement C3-mediated diseases and disorders, methods of inhibiting the activity of the complement Classical pathway (CP), Lectin pathway (LP), and/or Alternative pathway (AP), and methods of inhibiting the activity of choroidal-localized complement C3 are also provided.

### RELATED APPLICATION DISCLOSURE

This application is a continuation of U.S. application Ser. No. 17/669,398 filed Feb. 11, 2022, which claims the benefit of European Application No. PCT/EP2021/053526 filed Feb. 12, 2021, each of which is hereby incorporated by reference herein in its entirety.

### SEQUENCE DISCLOSURE

This application includes, as part of its disclosure, a “Sequence Listing XML” pursuant to 37 C.F.R. § 1.831 (a) which is electronically submitted in XML file format in a file named “02-0558-US-2.xml”, created on Apr. 3, 2025, and having a size of 29,455 bytes, which is hereby incorporated by reference herein in its entirety.

### FIELD OF THE INVENTION

This disclosure relates to antigen-binding proteins targeting complement C3 and methods of treating complement C3-mediated diseases.

### BACKGROUND

A major challenge in the treatment of certain ocular diseases and disorders is delivery of a therapeutic molecule to the deep layers of the retina. Delivery is impeded by multiple factors, including multiple physical boundaries within the eye. These boundaries include the corneal and conjunctival epithelium, blood-aqueous barriers (BAB), and blood-retinal barriers (BRB), such as capillary endothelial cells (inner BRB) and retinal pigment epithelial cells (RPE cells, outer BRB) (see, e.g., Jiang et al. Int J Ophthalmol. 2018; 11 (6): 1038-1044). Ocular diseases where delivery to the retina is particularly important are complement-mediated diseases, such as geographic atrophy (GA).

Geographic atrophy (GA) is an advanced form of age-related macular degeneration (AMD) characterized by loss of the retinal pigment epithelium and photoreceptors in the macula. Irreversible visual acuity loss occurs once GA involves the central fovea. Patients with earlier stages of GA typically experience visual function deficits even before visual acuity is affected.

The underlying pathophysiology of geographic atrophy is not completely understood; however, dysregulation of complement activity is thought to be a contributing factor. Several complement activation products, including C3a, C5a, C5b-9 and complement factor H (CFH) have shown elevated levels in vitreous samples, Bruch's membrane, and other parts of the choroid of GA patients compared with controls. In addition, complement inhibitors like CD59 a membrane-bound inhibitor of membrane attack complex (MAC) formation and membrane cofactor protein (MCP) a membrane-bound complement regulator that has cofactor activity for complement factor I (CFI) have been reported at reduced levels in GA.

At present, there are no approved treatments for GA. Multiple investigational approaches targeting the complement pathway have been explored, but none have yet been approved nor proven to be effective. Some examples of such approaches include eculizumab/SOLIRIS (Alexion), LFG-316 (Novartis/MorphoSys), ARC-1905 (Ophthotech), POT-4 (AL-78898A; Alcon) and lampalizumab (FCFD45142).

More recently, the findings from an APL-2 phase II clinical trial (Clinical Trial NCT02503332, “Study of APL-2 Therapy in Patients Geographic Atrophy (FILLY)”) further implicate the complement pathway in the pathogenesis of GA and show a positive treatment effect in reducing GA progression through complement inhibition. These results also suggest that APL-2 inhibition of the complement cascade centrally at C3 (which is the convergence of all complement pathways; see FIG. 1) may have the potential to treat GA more effectively than is possible with inhibitors that lead to partial inhibition of the complement pathways. Nevertheless, the reduction of lesion growth in GA obtained with APL-2 is still modest. APL-2 has features which may limit its effectiveness. APL-2, a pegylated derivative of the cyclic tridecapeptide compstatin (inhibitor of complement component C3) has a large molecular weight equivalent of 350 kDa and a hydrodynamic radius of about 7.8 nm, making it difficult to penetrate deeply into the retina. APL-2 only has an effective duration of 1 month, possibly due to a low concentration of 3.5 mM. APL-2 is also a PEGylated molecule, which increases its viscosity and may make it difficult to inject into the eye. Accordingly, there is a need for reducing GA progression more efficiently.

One major challenge in the treatment of GA is that the observed dysregulation of complement activity occurs in the deeper layers of the retina. We postulate that better penetration into disease-relevant retinal tissues (i.e. retinal pigment epithelium (RPE), Bruch's membrane, and other parts of the choroid) may be necessary to achieve greater reduction of lesion growth in GA. For this a small antibody fragment poses several advantages as compared to other biologics and antibodies. Small antibody formats may allow: 1) better intraocular penetration into relevant retinal tissues; and 2) more drug product per mg or mL delivered via intravitreal injection.

### SUMMARY

The present disclosure provides antigen binding proteins with specificity to complement C3.

In one aspect, the disclosure provides an antigen binding protein or fragment thereof which binds an epitope on complement C3, wherein the antigen binding protein or fragment thereof is capable of inhibiting the pathways of complement activation, including the Classical pathway (CP), the Lectin pathway (LP), and the Alternative pathway (AP).

In certain embodiments, the antigen binding protein or fragment thereof is capable of binding complement C3 and C3b.

In certain embodiments, the antigen binding protein or fragment thereof is capable of binding an epitope on complement C3, wherein such binding prevents the formation of C3 convertase.

In certain embodiments, the antigen binding protein or fragment thereof is capable of competing with one or more antigen binding proteins, including M0122, M0123, M0124, M0228, and M0251.

In certain embodiments, the antigen binding protein or fragment thereof comprises a single-chain variable fragment (scFv), a Fab fragment, a Fab′ fragment, a Fv fragment, a diabody, a small antibody mimetic or a single domain antibody, such as e.g., a sdAb, a sdFv, a nanobody, a V-Nar or a VHH. In preferred embodiments, the antigen binding protein or fragment thereof comprises a scFv or a VHH.

In certain embodiments, the antigen binding protein or fragment thereof comprises a CDR-H3 having at least 80% similarity to a sequence of the group consisting of SEQ ID NO: 3, SEQ ID NO: 6, SEQ ID NO: 9, SEQ ID NO: 15, and SEQ ID NO: 21.

In certain embodiments, the antigen binding protein or fragment thereof comprises a CDR-H3 having at least 80% identity to a sequence of the group consisting of SEQ ID NO: 3, SEQ ID NO: 6, SEQ ID NO: 9, SEQ ID NO: 15, and SEQ ID NO: 21.

In certain embodiments, the antigen binding protein or fragment thereof comprises a variable heavy chain (VH), and a variable light chain (VL), wherein the VH comprises a CDR-H1 sequence selected from the group consisting of SEQ ID NO: 1, 4, 7, 13, and 19, a CDR-H2 sequence selected from the group consisting of SEQ ID NO: 2, 5, 8, 14, and 20, a CDR-H3 sequence selected from the group consisting of SEQ ID NO: 3, 6, 9, 15, and 21; and wherein the VL comprises a CDR-L1 sequence selected from the group consisting of SEQ ID NO: 10, 16, and 22, a CDR-L2 sequence selected from the group consisting of SEQ ID NO: 11, 17, and 23, and a CDR-L3 sequence selected from the group consisting of SEQ ID NO: 12, 18, and 24.

In certain embodiments, the VH has at least 80% similarity to a sequence of the group consisting of SEQ ID NO: 25, 26, 27, 29, and 31, and/or the VL has at least 80% similarity to a sequence of the group consisting of SEQ ID NO: 28, 30, and 32.

In certain embodiments, the VH has at least 80% identity to a sequence of the group consisting of SEQ ID NO: 25, 26, 27, 29, and 31, and/or the VL has at least 80% similarity to a sequence of the group consisting of SEQ ID NO: 28, 30, and 32.

In certain embodiments, the antigen binding protein or fragment thereof comprises a VH and a VL, wherein the VH comprises a CDR-H1 sequence of SEQ ID NO: 7, a CDR-H2 sequence of SEQ ID NO: 8, and a CDR-H3 sequence of SEQ ID NO: 9; and wherein the VL comprises a CDR-L1 sequence of SEQ ID NO: 10, a CDR-L2 sequence of SEQ ID NO: 11, and a CDR-L3 sequence of SEQ ID NO: 12.

In certain embodiments, the VH comprises the amino acid sequence of SEQ ID NO: 27 and the VL comprises the amino acid sequence of SEQ ID NO: 28.

In certain embodiments, the antigen binding protein or fragment thereof comprises a VH and a VL, wherein the VH comprises a CDR-H1 sequence of SEQ ID NO: 13, a CDR-H2 sequence of SEQ ID NO: 14, and a CDR-H3 sequence of SEQ ID NO: 15; and wherein the VL comprises a CDR-L1 sequence of SEQ ID NO: 16, a CDR-L2 sequence of SEQ ID NO: 17, and a CDR-L3 sequence of SEQ ID NO: 18.

In certain embodiments, the VH comprises the amino acid sequence of SEQ ID NO: 29 and the VL comprises the amino acid sequence of SEQ ID NO: 30.

In certain embodiments, the antigen binding protein or fragment thereof comprises a VH and a VL, wherein the VH comprises a CDR-H1 sequence of SEQ ID NO: 19, a CDR-H2 sequence of SEQ ID NO: 20, and a CDR-H3 sequence of SEQ ID NO: 21; and wherein the VL comprises a CDR-L1 sequence of SEQ ID NO: 22, a CDR-L2 sequence of SEQ ID NO: 23, and a CDR-L3 sequence of SEQ ID NO: 24.

In certain embodiments, the VH comprises the amino acid sequence of SEQ ID NO: 31 and the VL comprises the amino acid sequence of SEQ ID NO: 32.

In certain embodiments, the antigen binding protein or fragment thereof comprises a VHH domain, wherein the VHH domain comprises a CDR-H1 sequence of SEQ ID NO: 1, a CDR-H2 sequence of SEQ ID NO: 2, and a CDR-H3 sequence of SEQ ID NO: 3.

In certain embodiments, the VHH domain comprises the amino acid sequence of SEQ ID NO: 25.

In certain embodiments, the antigen binding protein or fragment thereof comprises a VHH domain, wherein the VHH domain comprises a CDR-H1 sequence of SEQ ID NO: 4, a CDR-H2 sequence of SEQ ID NO: 5, and a CDR-H3 sequence of SEQ ID NO: 6.

In certain embodiments, the VHH domain comprises the amino acid sequence of SEQ ID NO: 26.

In certain embodiments, the antigen binding protein or fragment thereof comprises a binding affinity for C3 and C3b of at least about 10⁻⁸ M. In certain embodiments, the antigen binding protein or fragment thereof comprises a binding affinity for C3 and C3b of about 10⁻⁹ M to about 10⁻¹⁴ M. In certain embodiments, the antigen binding protein or fragment thereof comprises a binding affinity for C3 and C3b of about 10⁻¹⁰ M to about 10⁻¹² M. In certain embodiments, the antigen binding protein or fragment thereof comprises approximately equivalent binding affinity for C3 and C3b. In certain embodiments, the binding affinity for C3 is within a factor of 10 of the binding affinity for C3b.

In certain embodiments, the antigen binding protein or fragment thereof comprises a binding affinity for C3a, iC3b, C4, C4b, C5, and/or C5b of about 10⁻⁴ M or weaker. In certain embodiments, the antigen binding protein or fragment thereof comprises weaker binding affinity for C3a, iC3b, C4, C4b, C5, and/or C5b compared to the binding affinity for C3 and C3b. In certain embodiments, the antigen binding protein or fragment thereof comprises no binding affinity for C3a, iC3b, C4, C4b, C5, and/or C5b.

In certain embodiments, the antigen binding protein or fragment thereof is capable of inhibiting the activity of the CP, LP, and AP complement pathways by at least about 80%, at least about 85%, at least about 90%, or at least about 95%.

In certain embodiments, the antigen binding protein or fragment thereof is capable of equivalent or approximately equivalent inhibition of the activity of the CP, LP, and AP complement pathways. In certain embodiments, the inhibition of the activity of the CP, LP, and AP complement pathways is at least about 80%, at least about 85%, at least about 90%, or at least about 95%.

In certain embodiments, the activity of the CP, LP, and AP complement pathways is determined by measuring the level of erythrocyte hemolysis in the presence of antigen binding protein or fragment thereof compared to the level of erythrocyte hemolysis in the absence of antigen binding protein or fragment thereof.

In certain embodiments, the activity of the CP, LP, and AP complement pathways is determined by measuring Membrane Attack Complex (MAC) formation in the presence of antigen binding protein or fragment thereof compared to MAC formation in the absence of antigen binding protein or fragment thereof.

In certain embodiments, the antigen binding protein or fragment thereof is capable of inhibiting the activity of C3 convertase by at least about 80%, at least about 85%, at least about 90%, or at least about 95%.

In certain embodiments, the antigen binding protein or fragment thereof is capable of inhibiting the C3 convertase amplification loop.

In certain embodiments, the antigen binding protein or fragment thereof is capable of penetrating Bruch's membrane.

In certain embodiments, the antigen binding protein or fragment thereof is capable of inhibiting choroidal C3 activity.

In certain embodiments, the antigen binding protein or fragment thereof comprises a molecular weight of about 60 kDa or less. In certain embodiments, the antigen binding protein or fragment thereof comprises a molecular weight of about 20 kDa to about 30 kDa. In certain embodiments, the antigen binding protein or fragment thereof comprises a molecular weight of about 10 kDa to about 20 kDa. In certain embodiments, the antigen binding protein or fragment thereof comprises a molecular weight of about 25 kDa. In certain embodiments, the antigen binding protein or fragment thereof comprises a molecular weight of about 15 kDa.

In certain embodiments, the antigen binding protein or fragment thereof comprises cross-reactivity with cynomolgus C3.

In one aspect, the disclosure provides a pharmaceutical composition comprising the antigen binding protein or fragment thereof described above, and a pharmaceutically acceptable carrier. Thus, one aspect is the use of the binding protein of the invention in the preparation of a pharmaceutical composition for treating a complement C3-mediated disease or disorder in a subject.

In certain embodiments, the pharmaceutical composition comprises low viscosity.

In certain embodiments, the viscosity is between about 1 cP to about 50 cP. In certain embodiments, the viscosity is less than or equal to about 20 cP.

In one aspect, the disclosure provides an isolated nucleic acid molecule encoding the antigen binding protein or fragment thereof described above.

In another aspect, the disclosure provides an expression vector comprising the nucleic acid molecule described above.

In yet another aspect, the disclosure provides a host cell comprising the expression vector described above.

In yet another aspect, a method of manufacturing an antigen binding protein or fragment thereof as described above is provided, comprising
i) cultivating the host cell as described above under conditions allowing expression of the protein described herein; and,
ii) recovering the protein; and optionally
iii) further purifying and/or modifying and/or formulating the protein.

In one aspect, the disclosure provides a method for treating a complement C3-mediated disease or disorder in a subject, comprising administering to a subject in need thereof the antigen binding protein or fragment thereof described above. Thus, the disclosure also provides an antigen binding protein or fragment thereof as described above for use in a method of treating a complement C3-mediated disease or disorder. In certain embodiments, the antigen binding protein or fragment thereof is administered via topical, subconjunctival, intravitreal, retrobulbar, and/or intracameral administration.

In certain embodiments, the complement C3-mediated disease or disorder is selected from a group consisting age-related macular degeneration, geographic atrophy, neovascular glaucoma, diabetic retinopathy, retinopathy of prematurity, retrolental fibroplasia, autoimmune uveitis, chorioretinitis, retinitis, rheumatoid arthritis, psoriasis and atherosclerosis.

In one aspect, the disclosure provides a method of inhibiting the activity of the complement Classical pathway (CP), Lectin pathway (LP), and Alternative pathway (AP), the method comprising contacting complement C3 with an antigen binding protein or fragment thereof which binds an epitope on complement C3. Thus, the disclosure provides an antigen binding protein or fragment thereof as described herein for use in a method of treating a complement C3-mediated disease or disorder, by inhibiting the activity of the complement Classical pathway (CP), Lectin pathway (LP), and Alternative pathway (AP). The disclosure also provides an antigen binding protein or fragment thereof as described above for use in a method of treating a complement C3-mediated disease or disorder, by inhibiting the activity of choroidal-localized complement C3.

In one aspect, the disclosure provides a method of inhibiting the activity of choroidal-localized complement C3, the method comprising intraocular administration of an antigen binding protein or fragment thereof which binds an epitope on complement C3.

In certain embodiments of the methods described herein, the antigen binding protein or fragment thereof is capable of binding complement C3 and C3b.

In certain embodiments, the antigen binding protein or fragment thereof is capable of binding an epitope on complement C3, wherein such binding prevents the formation of C3 convertase.

In certain embodiments, the antigen binding protein or fragment thereof is capable of competing with one or more antigen binding proteins, including M0122, M0123, M0124, M0228, and M0251.

In certain embodiments, the antigen binding protein or fragment thereof comprises a single-chain variable fragment (scFv), a Fab fragment, or a VHH.

In certain embodiments, the antigen binding protein or fragment thereof comprises a CDR-H3 having at least 80% similarity to a sequence of the group consisting of SEQ ID NO: 3, SEQ ID NO: 6, SEQ ID NO: 9, SEQ ID NO: 15, and SEQ ID NO: 21.

In certain embodiments, the antigen binding protein or fragment thereof comprises a CDR-H3 having at least 80% identity to a sequence of the group consisting of SEQ ID NO: 3, SEQ ID NO: 6, SEQ ID NO: 9, SEQ ID NO: 15, and SEQ ID NO: 21.

In certain embodiments, the antigen binding protein or fragment thereof comprises a variable heavy chain (VH), and a variable light chain (VL), wherein the VH comprises a CDR-H1 sequence selected from the group consisting of SEQ ID NO: 1, 4, 7, 13, and 19, a CDR-H2 sequence selected from the group consisting of SEQ ID NO: 2, 5, 8, 14, and 20, a CDR-H3 sequence selected from the group consisting of SEQ ID NO: 3, 6, 9, 15, and 21; and wherein the VL comprises a CDR-L1 sequence selected from the group consisting of SEQ ID NO: 10, 16, and 22, a CDR-L2 sequence selected from the group consisting of SEQ ID NO: 11, 17, and 23, and a CDR-L3 sequence selected from the group consisting of SEQ ID NO: 12, 18, and 24.

In certain embodiments, the VH has at least 80% similarity to a sequence of the group consisting of SEQ ID NO: 25, 26, 27, 29, and 31, and/or the VL has at least 80% similarity to a sequence of the group consisting of SEQ ID NO: 28, 30, and 32.

In certain embodiments, the VH has at least 80% identity to a sequence of the group consisting of SEQ ID NO: 25, 26, 27, 29, and 31, and/or the VL has at least 80% identity to a sequence of the group consisting of SEQ ID NO: 28, 30, and 32.

In certain embodiments, the antigen binding protein or fragment thereof is capable of penetrating Bruch's membrane.

In certain embodiments, the antigen binding protein or fragment thereof is capable of inhibiting choroidal C3 activity.

In certain embodiments, antigen binding protein or fragment thereof comprises a molecular weight of about 60 kDa or less, such as about 50 kDa or less, about 40 kDa or less, about 35 kDa or less, about 30 kDa or less, about 25 kDa or less, about 20 kDa or less, about 15 kDa or less. In certain embodiments, the antigen binding protein or fragment thereof comprises a molecular weight of about 20 kDa to about 30 kDa. In certain embodiments, the antigen binding protein or fragment thereof comprises a molecular weight of about 10 kDa to about 20 kDa. In certain embodiments, the antigen binding protein or fragment thereof comprises a molecular weight of about 25 kDa. In certain embodiments, the antigen binding protein or fragment thereof comprises a molecular weight of about 15 kDa.

On one aspect, the disclosure provides a method of detecting one or both of C3 and C3b in a biological sample comprising
(a) contacting the sample with at least one antigen binding protein or fragment thereof as described above;
(b) permitting formation of complexes between one or both of C3 and C3b and the antigen binding protein or fragment thereof in the sample; and
(c) detecting said antigen binding protein or fragment thereof. In preferred embodiments, the antigen binding protein or fragment thereof is capable of binding complement C3 and C3b.

In one embodiment, the antigen binding protein or fragment thereof is detected by a detectable signal.

In one embodiment, the antigen binding protein or fragment thereof is detected by ELISA, immunocytochemistry (ICC), immunohistochemistry (IHC), Western Blotting and/or Flow cytometry.

The biological sample may be a tissue sample, e.g., retinal tissue of a human subject, such as a fixed tissue sample. The fixed tissue sample may be a formalin-fixed and paraffin-embedded tissue sample.

In one aspect, a kit for detecting C3 is provided, comprising the antigen binding protein or fragment thereof as described above, and instructions for use.

### BRIEF DESCRIPTION OF THE DRAWINGS

The foregoing and other features and advantages of the present invention will be more fully understood from the following detailed description of illustrative embodiments taken in conjunction with the accompanying drawings. Copies of this patent or patent application publication with color drawing(s) will be provided by the Office upon request and payment of the necessary fee.

FIG. 1 depicts three complement pathways: classical (CP), lectin (LP) and alternative (AP) pathways, which converge at C3.

FIG. 2 depicts the process of generating an anti-C3 antibody library.

FIG. 3A-FIG. 3B depicts an ELISA assay which confirms excellent immune response against C3 in rabbit and llama. FIG. 3A depicts an ELISA assay testing the C3 protein isolated from human plasma. FIG. 3B depicts an ELISA assay testing the presence of anti-C3 antibodies in the sera of rabbit (top panel) and llama (bottom panel) injected with the isolated human C3 shown in FIG. 3A.

FIG. 4A depicts a summary of anti-C3 antibody libraries and FIG. 4B shows CDR-H3 amino acid length diversity.

FIG. 5 depicts the process of screening for anti-C3 antibodies.

FIG. 6 depicts a screen of candidate antibodies targeting C3 for their ability to inhibit all three complement pathways in human serum. Each antibody was used at a concentration of 2 μM.

FIG. 7A-FIG. 7D demonstrate that the four anti-C3 antibodies which inhibit all three complement pathways recognize three different epitopes on C3. FIG. 7A depicts a competition assay which shows there is no competition between M0122 with each of the other 3 anti-C3 antibodies. FIG. 7B depicts a competition assay which shows there is no competition between M0124 with each of the other 3 anti-C3 antibodies. FIG. 7C depicts a competition assay which shows there is competition between M0228 and M0251, but not M0124 and M0122. FIG. 7D depicts a competition assay which shows there is competition between M0123 and M0251, and M0123 and M0228, but not M0124 and M0122.

FIG. 8A-FIG. 8B depict that M0122, M0124 and M0228 bind both C3 and C3b directly. FIG. 8A depicts an ELISA assay which demonstrates M0122, M0124 and M0228 bind to C3 directly. FIG. 8B depicts an ELISA assay which demonstrates M0122, M0124 and M0228 bind to C3b directly.

FIG. 9A-FIG. 9B depict that M0122, M0124 and M0228 potently inhibit classical and alternative pathways. FIG. 9A depicts that M0122, M0124 and M0228 potently inhibit classical pathway. FIG. 9B depicts that M0122, M0124 and M0228 potently inhibit alternative pathways.

FIG. 10 depicts the affinities parameters of M0122, M0124 and M0228.

FIG. 11 depicts the schematic of the anatomic structure of the retina and the choroid, including the Bruch's membrane. The anti-C3 scFv antibodies of the disclosure are depicted as being capable of penetrating the Bruch's membrane and entering deeper into the choroid, while a comparative C3-binding therapeutic, APL-2, cannot penetrate the Bruch's membrane. The same principle applies to other antigen binding protein formats of the invention.

FIG. 12 depicts the negative relation between hydrodynamic radii and permeability, with complement binding therapeutics shown compared to an scFv of the disclosure.

FIG. 13A and FIG. 13B depict a comparison of an scFv and an APL-2 surrogate for penetrating a Bruch's membrane. FIG. 14A shows a barium iodide staining (PEG), FIG. 13B shows a Coomassie staining (protein). The APL2-surrogate comprises one APL-1 moiety on 40 kDa linear PEG. SC—Sample chamber, DC—Diffusate chamber, LC—Loading control (initial concentration in SC).

FIG. 14A-FIG. 14C depict that M0122, M0124 and M0251 potently inhibit classical, alternative, and lectin pathways in cyno serum. FIG. 14A depicts that M0122, M0124 and M0251 potently inhibit all three pathways. Each antibody was used at a concentration of 2 μM. FIG. 14B depicts that M0122, M0124 and M0251 potently inhibit classical pathway. FIG. 14C depicts that M0122, M0124 and M0251 potently inhibit alternative pathways.

FIG. 15A-FIG. 15B depict that M0122, M0124, and M0251 bind cyno C3. FIG. 15A depicts M0122, M0124. FIG. 15B depicts M0251.

FIG. 16 depict that M0122, M0123 and M0124 potently inhibit lectin pathway.

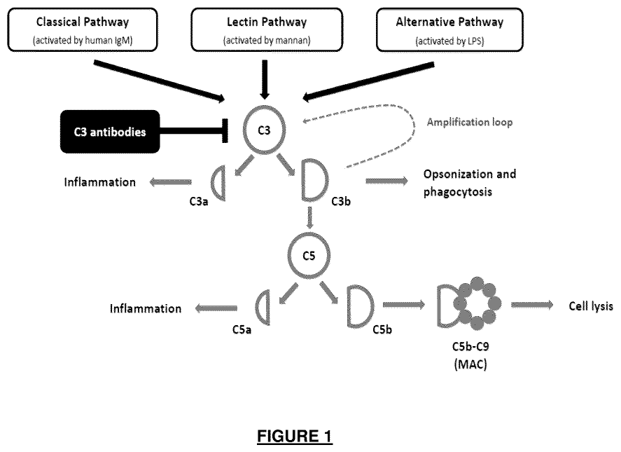

### DETAILED DESCRIPTION

Antigen binding proteins having binding specificity for complement C3 and the complement C3 cleavage product, C3b, are provided. Methods for treating or preventing complement C3-mediated diseases and disorders are also provided.

In certain aspects, antigen binding proteins described herein are capable of inhibiting the complement Classical pathway (CP), Lectin pathway (LP), and Alternative pathway (AP). The antigen binding proteins described herein may inhibit all three pathways simultaneously. The antigen binding proteins described herein may inhibit all three pathways in the choroid of the eye.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

### Antigen Binding Proteins

As used herein, the term “antibody” or “antigen binding protein” refers to an immunoglobulin molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)₂ fragments, Fab′ fragments, Fv fragments, recombinant IgG (rIgG) fragments, single chain variable fragments (scFv) and single domain antibodies (e.g., sdAb, sdFv, nanobody, VHH) fragments. The term “antibody” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof. The term “antibody fragments” as used herein includes artificial proteins designed to selectively bind antigens, i.e., antibody mimetics. Typically, one or more CDRs are grafted to non-Ig scaffolds, thereby mimicking the CDR conformations from the parental antibody. Non-limiting examples of such antibody mimetics include fluctuation-regulated affinity proteins (FLAPs), monobodies and affimers. Antibody mimetics may comprise one, two, three, four, five or six of the CDRs as described herein.

As used herein, a “Fab fragment” is an antibody fragment comprising a light chain fragment comprising a variable light (VL) domain and a constant domain of the light chain (CL), and variable heavy (VH) domain and a first constant domain (CH1) of the heavy chain. A Fab fragment generally has a molecular weight of about 50 kDa and a hydrodynamic radius of about 3.0 nm.

As used herein, a “single-chain variable fragment” (scFv) is an antigen binding protein comprising a heavy chain variable domain (VH) linked to a light chain variable domain (VL). The VH and VL domains of the scFv are linked via any appropriate art recognized linker. Such linkers include, but are not limited to, repeated GGGGS (SEQ ID NO: 33) amino acid sequences or variants thereof. The scFv is generally free of antibody constant domain regions, although an scFv of the disclosure may be linked or attached to antibody constant domain regions (e.g., antibody Fc domain) to alter various properties of the scFv, including, but not limited to, increased serum or tissue half-life. An scFv generally has a molecular weight of about 25 kDa and a hydrodynamic radius of about 2.5 nm.

As used herein, a “VHH”, “nanobody”, or “heavy-chain only antibody” is an antigen binding protein comprising a single heavy chain variable domain derived from the species of the Camelidae family, which includes camels, llama, alpaca. A VHH generally has a molecular weight of about 15 kDa.

As used herein, the term “complementarity determining region” or “CDR” refers to non-contiguous sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FRs” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4). With regards to VHH antibodies, there are only three heavy chain CDRs and no light chain CDRs.

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309 (3): 657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Variants of the antibodies provided herein may be generated by introducing deletions, substitutions, additions, and/or modifications to the framework and/or to the CDRs. The antibody variants can then be tested for the desired function using methods described herein. Any combination(s) of deletions, substitutions, additions, modifications and insertions can be made to the antigen binding protein or fragment thereof, provided that the generated variant possesses the desired characteristics for which it can be screened using appropriate methods.

As used herein, a “conservative substitution” refers to a modification that maintains the functional properties of the parental antibody. For example, conservative amino acid substitutions include those in which the amino acid residue is replaced with an amino acid residue having similar properties. For example, substituting alanine (A) by valine (V); substituting arginine (R) by lysine (K); substituting asparagine (N) by glutamine (Q); substituting aspartic acid (D) by glutamic acid (E); substituting cysteine (C) by serine(S); substituting glutamic acid (E) by aspartic acid (D); substituting glycine (G) by alanine (A); substituting histidine (H) by arginine (R) or lysine (K); substituting isoleucine (I) by leucine (L); substituting methionine (M) by leucine (L); substituting phenylalanine (F) by tyrosine (Y); substituting serine(S) by threonine (T); substituting tryptophan (W) by tyrosine (Y); substituting phenylalanine (F) by tryptophan (W); and/or substituting valine (V) by leucine (L) and vice versa.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the known schemes. Likewise, unless otherwise specified, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the Kabat, Chothia, Contact, IMGT, or AHo method. In other cases, the particular amino acid sequence of a CDR or FR is given. CDR and FR numbering is further described in Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309 (3): 657-70, (AHo numbering scheme).

The terms “compete” or “cross-compete” are used interchangeably herein to refer to the ability of an antibody molecule to interfere with binding of an antibody molecule, e.g., the antigen binding proteins as described herein, to a target, e.g., human C3 and/or C3b. The interference with binding can be direct or indirect (e.g., through an allosteric modulation of the antigen binding molecule or the target). The extent to which antigen binding molecule is able to interfere with the binding of another antigen binding molecule to the target, and therefore whether it can be said to compete, can be determined using a competition binding assay, for example, a FACS assay, an ELISA or BIACORE assay. In some embodiments, a competition binding assay is a quantitative competition assay. In some embodiments, a first antigen binding molecule is said to compete for binding to the target with a second antigen binding molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

As used herein, the term “affinity” refers to the strength of the interaction between an antibody's antigen binding site and the epitope to which it binds. As readily understood by those skilled in the art, an antibody or antigen binding protein affinity may be reported as a dissociation constant (K_{D}) in molarity (M). The antibodies of the disclosure may have K_{D} values in the range of 10⁻⁵ to 10⁻¹² M. High affinity antibodies have K_{D} values of 10⁻⁹ M (1 nanomolar, nM) and lower. For example, a high affinity antibody may have K_{D} value in the range of about 1 nM to about 0.01 nM. A high affinity antibody may have K_{D} value of about 1 nM, about 0.9 nM, about 0.8 nM, about 0.7 nM, about 0.6 nM, about 0.5 nM, about 0.4 nM, about 0.3 nM, about 0.2 nM, or about 0.1 nM. Very high affinity antibodies have K_{D} values of 10⁻¹² M (1 picomolar, pM) and lower. Weak, or low, affinity antibodies may have K_{D} values in the range of 10⁻¹ to 10⁻⁴ M. Low affinity antibodies may have K_{D} values of 10⁻⁴ M and higher, such as 10⁻⁴ M, 10⁻³ M, 10⁻² M, or 10⁻¹ M.

In certain embodiments, the antigen binding proteins of the disclosure have a binding affinity for C3 and C3b of about 10⁻⁸ M to about 10⁻¹⁴ M. In certain embodiments, the antigen binding proteins of the disclosure have a binding affinity for C3 and C3b of about 10⁻¹⁰ M to about 10⁻¹² M. In certain embodiments, the antigen binding proteins of the disclosure have a binding affinity for C3 and C3b of at least about 10⁻⁸ M, at least about 10⁻⁹ M, at least about 10⁻¹⁰ M, at least about 10⁻¹¹ M, or at least about 10⁻¹² M.

In certain embodiments, the antigen binding protein or fragment thereof comprises approximately equivalent binding affinity for C3 and C3b. For example, but in no way limiting, the antigen binding protein or fragment thereof can comprise a binding affinity for C3 of about 10⁻¹⁰ M and a binding affinity for C3b of about 10⁻¹⁰ M. In certain embodiments, the antigen binding protein or fragment thereof comprises a binding affinity for C3 of about 10⁻¹¹ M and a binding affinity for C3b of about 10⁻¹¹ M. In certain embodiments, the antigen binding protein or fragment thereof comprises a binding affinity for C3 of about 10⁻¹² M and a binding affinity for C3b of about 10⁻¹² M.

In certain embodiments, the binding affinity for C3 is within a factor of 10 of the binding affinity for C3b. For example, but in no way limiting, the antigen binding protein or fragment thereof can comprise a binding affinity for C3 of about 10⁻¹⁰ M and a binding affinity for C3b of about 10⁻¹¹ M. In certain embodiments, the antigen binding protein or fragment thereof comprises a binding affinity for C3 of about 10⁻¹¹ M and a binding affinity for C3b of about 10⁻¹² M.

In certain embodiments, the antigen binding protein or fragment thereof comprises cross-reactivity with cynomolgus C3. Cynomolgus (Macaca fascicularis) C3 is 95.1% identical to human C3 and cross-reactivity allows for the preclinical and toxicology testing of the antigen binding proteins of the disclosure in a relevant animal model.

For avoidance of doubt, and unless otherwise indicated, C3 as used herein refers to human of complement component 3 of UniProt P01024 and the nucleic acid sequence encoding that protein. C3b is derived from native C3 and is the larger of two elements formed by the cleavage of C3.

In certain embodiments, the antigen binding proteins of the disclosure are monovalent and bind human C3 and C3b with a K_{D} of about 200 nM or lower as measured with biolayer interferometry (BLI). In certain embodiments, the K_{D} is about 200 pM or lower, such as about 100 pM, about 10 pM, about 1 pM, or about 0.1 pM.

The ability of an antigen binding domain to bind to a specific antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)).

### Anti-Complement C3 Antigen Binding Proteins

In one aspect, the disclosure provides antigen binding proteins with binding specificity to complement C3 protein. In certain embodiments, the anti-C3 antigen binding proteins are scFvs, Fab fragments, or VHH.

Exemplary anti-C3 antigen binding protein CDRs are recited below in Table 1. Exemplary anti-C3 antigen binding protein variable heavy and variable light domains are recited below in Table 2. The exemplary anti-C3 antigen binding proteins recited below were generated through the immunization of rabbits and llamas with human C3 protein isolated from human plasma. The exemplary VH and VL domains of M0122, M0123, and M0124 were derived from rabbits immunized with human C3 protein and are wild-type rabbit sequences. The exemplary VHH domains of M0228 and M0251 were derived from llamas immunized with human C3 protein and are wild-type llama sequences.

TABLE 1
Anti-C3 antigen binding protein CDR sequences.

| SEQ ID NO: | Sequence | Note |
|---|---|---|
| 1 | DYTMG | M0228_CDR-H1 |
| 2 | AINWRGSSTYYADSVKG | M0228_CDR-H2 |
| 3 | QVSPYVELTATAAY | M0228_CDR-H3 |
| 4 | NWAMG | M0251_CDR-H1 |
| 5 | AIRWSVGTTNYRDSVKG | M0251_CDR-H2 |
| 6 | GTPFVLARINGYDY | M0251_CDR-H3 |
| 7 | NYAMN | M0122_CDR-H1 |
| 8 | IINTDGNTNYASWAKG | M0122_CDR-H2 |
| 9 | AVGYHHHALDP | M0122_CDR-H3 |
| 10 | TLSSAHKTYTID | M0122_CDR-L1 |
| 11 | LKSDGSYTKGT | M0122_CDR-L2 |
| 12 | GTDYGGGYV | M0122_CDR-L3 |
| 13 | SYHMS | M0123_CDR-H1 |
| 14 | IIYTDGNTDYANWAKG | M0123_CDR-H2 |
| 15 | RGYADYGYTFNL | M0123_CDR-H3 |
| 16 | TADTLSRNYAS | M0123_CDR-L1 |
| 17 | RDTSRPS | M0123_CDR-L2 |
| 18 | ATGDGSGSSYQFV | M0123_CDR-L3 |
| 19 | RYWMN | M0124_CDR-H1 |
| 20 | YITTNDKTYYANWAKG | M0124_CDR-H2 |
| 21 | RSSGAYDI | M0124_CDR-H3 |
| 22 | TLSSAHKTYYIE | M0124_CDR-L1 |
| 23 | LKSDGTYTKGT | M0124_CDR-L2 |
| 24 | GVTGGNVYV | M0124_CDR-L3 |

TABLE 2
Anti-C3 antigen binding protein VH/VL sequences.

| SEQ ID NO: | Sequence | Note |
|---|---|---|
| 25 | EVQLVESGGGLVQAGGSLRLSCAAS | M0228_VHH |
| | GRTINDYTMGWFRQAPGKDREFVSA | |
| | INWRGSSTYYADSVKGRFTISRDNAK | |
| | KTIYLQMNLLKPEDTAVYYCARQVS | |
| | PYVELTATAAYWGQGTQVTVSS | |
| 26 | EVQLVESGGGLVQAGGSLRLSCVAS | M0251_VHH |
| | GHTFGNWAMGWFRQAPGKEREFVG | |
| | AIRWSVGTTNYRDSVKGRFAISRDN | |
| | ARNTVYLQMNRLKPEDTAVYYCAA | |
| | GTPFVLARINGYDYWGQGTQVTVSS | |
| 27 | QSVKESGGRLVTPGTPLTLTCTVSGF | M0122_VH |
| | SLYNYAMNWVRQAPGKGLEWIGIIN | |
| | TDGNTNYASWAKGRFTISTTSSTTVD | |
| | LKITSPTTEDTATYFCPRAVGYHHHA | |
| | LDPWGPGTLVTVSS | |
| 28 | ELVLTQSPSVSAALGASAKLTCTLSS | M0122_VL |
| | AHKTYTIDWYQQQQGEAPRYLMQL | |
| | KSDGSYTKGTGVPDRFSGSSSGADR | |
| | YLIIPSVQADDEADYYCGTDYGGGY | |
| | VFGGGTQLTVTG | |
| 29 | QSVKESEGRLVTPGTPLTLTCTASGF | M0123_VH |
| | TIGSYHMSWVRQAPGKGLEWIGIIYT | |
| | DGNTDYANWAKGRFTISKTSTTMDL | |
| | KMTSLTAADTATYFCARRGYADYG | |
| | YTFNLWGQGTLVTISS | |
| 30 | ELVLTQPASVQVNLGQTVSLTCTAD | M0123_VL |
| | TLSRNYASWYQQKPGQAPVLLIYRD | |
| | TSRPSGVPDRFSGSSSGNTATLTISGA | |
| | QAGDEADYYCATGDGSGSSYQFVFG | |
| | GGTQLTVTG | |
| 31 | QSVKESGGRLVTPGTPLTLTCTVSGI | M0124_VH |
| | DLSRYWMNWVRQAPGKGLEWIGYI | |
| | TTNDKTYYANWAKGRYTISKTSSTT | |
| | VDLKMTSLTTEDTATYFCARRSSGA | |
| | YDIWGPGTLVTISS | |
| 32 | QPVLTQSPSASATLGASAKLTCTLSS | M0124_VL |
| | AHKTYYIEWYQQQQGEAPRYLMQL | |
| | KSDGTYTKGTGVPDRFSGSSSGADR | |
| | YLIISSVQAEDEADYICGVTGGNVYV | |
| | FGGGTQLTVTG | |

In certain embodiments, the anti-C3 antigen binding proteins of the disclosure comprise at least about 80%, at least about 80%, at least about 85%, at least about 90%, at least about 95%, at least about 96%, at least about 97%, at least about 98%, at least about 99%, or 100% sequence similarity or identity to any of the sequences of Table 1 or Table 2.

In certain embodiments, the anti-C3 antigen binding proteins of the disclosure are selected for their ability to inhibit one or more complement pathways, the Classical pathway, the Alternative pathway, and the Lectin pathway. In certain embodiments, the anti-C3 antigen binding proteins of the disclosure are selected for their ability to inhibit all three complement pathways, the Classical pathway, the Alternative pathway, and the Lectin pathway. In certain embodiments, the anti-C3 antigen binding proteins of the disclosure are capable of inhibiting all three complement pathways in the eye. In certain embodiments, the anti-C3 antigen binding proteins of the disclosure are capable of inhibiting all three complement pathways in the choroid region of the eye. The choroid region is a layer containing blood vessels that lines the back of the eye and is located between the retina and the sclera. The choroid region is divided into four layers, Haller's layer, Sattler's layer, the Choriocapillaris, and Bruch's membrane. Bruch's membrane, also known as the vitreous lamina, is the innermost layer of the choroid and is adjacent to the retinal pigment epithelium (RPE). In certain embodiments, the anti-C3 antigen binding proteins of the disclosure are capable of penetrating or diffusing across Bruch's membrane and entering the other layers of the choroid, such as, but not limited to, the Choriocapillaris.

The retina has substantial physical barriers that may prevent large molecules, such as full-length immunoglobulins, to penetrate to deeper layers which may result in reduced therapeutic effects (Jackson et al. Invest Ophthalmol Vis Sci. 2003; 44 (5): 2141-6). Smaller antibody derivatives may in contrast penetrate deeper into the retina. Exemplary antibody derivates having a molecular weight of about 60 kDa or lower are antibody fragments, including, but not limited to, a Fab, a Fab′ fragment, a scFab, an scFv, a Fv fragment, a nanobody, a VHH, a dAb, a V-Nar, sdAb, a sdFv, and bispecific and bivalent antibodies such as a single-chain diabody (scDb), or a DART. In certain embodiments, the anti-C3 antigen binding proteins of the disclosure have a molecular weight of about 60 kDa or lower, for example, about 55 kDa, about 50 kDa, about 45 kDa, about 40 kDa, about 35 kDa, about 30 kDa, about 25 kDa, about 20 kDa, about 15 kDa, or lower.

In certain embodiments, the anti-C3 antigen binding proteins of the disclosure are capable of penetrating or diffusing across Bruch's membrane in part due to their size, which is sufficiently low to facilitate penetration. In certain embodiments, the size of the antigen binding proteins of the disclosure are measured by molecular weight. In certain embodiments, the antigen binding proteins of the disclosure have a molecular weight that is less than about 60 kDa. In certain embodiments, the antigen binding proteins of the disclosure are about 20 kDa to about 30 kDa or about 10 kDa to about 20 kDa. In certain embodiments, the antigen binding proteins of the disclosure are about 25 kDa. In certain embodiments, the antigen binding proteins of the disclosure are about 15 kDa. In certain embodiments, the size of the antigen binding proteins of the disclosure are measured by their hydrodynamic radius. In certain embodiments, the antigen binding proteins of the disclosure have a hydrodynamic radius of less than or equal to about 3.0 nm. In certain embodiments, the antigen binding proteins of the disclosure have a hydrodynamic radius of less than or equal to about 2.5 nm. In certain embodiments, the antigen binding proteins of the disclosure have a hydrodynamic radius of less than or equal to about 2.0 nm.

In certain embodiments, the anti-C3 antigen binding proteins of the disclosure are capable of competing with one or more antigen binding proteins, including M0122, M0123, M0124, M0228, and M0251. Antibody competition may be measured by any assay known in the art. In certain embodiments, one antibody may be labelled with a marker, such as biotin, and incubated together with other anti-C3 antibodies in a C3 binding ELISA. Usually, when a competing antigen binding protein is present in excess, it will reduce specific binding of the antigen binding protein or fragment thereof as described herein to C3 and/or C3b, i.e. it cross-blocks binding, by at least 40-45%, 45-50%, 50-55%, 55-60%, 60-65%, 65-70%, 70-75% or 75% or more. In certain embodiments, binding of the antigen binding protein or fragment thereof described herein in presence of the competing antigen binding protein is reduced by at least 80-85%, 85-90%, 90-95%, 95-97%, or 97% or more.

Complement C3 is a large protein composed of 13 different domains and a molecular size of 185 kilodaltons. During complement activation, C3 undergoes proteolytic cleavage and structural modifications at different sites. The C3 derived fragments exert different effector functions and form convertases that fuel amplification loops to the three complement pathways. The Classical pathway and Lectin pathway C3 convertase, C4bC2a, cleaves full length C3 into C3b and the anaphylatoxin C3a. The Alternative pathway also generates C3b and C3a, but utilizes the Alternative pathway C3 convertase, C3bBb. Furthermore, additional C3 degradation products may be generated in the complement pathways. Complement Factor I (CFI) is a plasma serine protease that is able to permanently inactivate C3b to iC3b. iC3b then is cleaved into further fragments (C3dg and C3c) by CFI. An additional C3 proteolytic product, C3d, binds complement receptor 2 (CR2) and may play an important role in the cell-cycle control of B cells. Along with the C3-derived protein products, the complement pathways include, but are not limited to, C1, C2, C4, C4b, C4a C5, C5b, C5a, C6, C7, C8, C9, C1q, C1r, C1s, Factor B, Factor D, Factor P, Factor H, Factor I, CD46 (MCP), CD55 (DAF), CD59 (MAC-IP), CR1 (CD35), CR2 (CD21), CR3, CR4, C3aR, C5aR1, C5aR2, CRIg, C4BP α-chain, C4BP β-chain, ficolin-1, mannose-binding lectin (MBL), MBL-associated serine protease-1 (MASP-1), and MBL-associated serine protease-2 (MASP-2). The complement pathway and various complement pathway components are described in further detail in Noris et al. Semin Nephrol. 2013; 33 (6): 479-492.

In certain embodiments, the disclosure provides anti-C3 antigen binding proteins capable of binding both C3 and C3b. In certain embodiments, the anti-C3 antigen binding proteins of the disclosure comprise a binding affinity for C3a, iC3b, C4, C4b, C5, and/or C5b that is weaker than the binding affinity for C3 and C3b. In certain embodiments, the anti-C3 antigen binding proteins of the disclosure comprise a binding affinity for C3a, iC3b, C4, C4b, C5, and/or C5b of about 10⁻⁴ M or weaker. In certain embodiments, the anti-C3 antigen binding proteins of the disclosure comprise no binding affinity for C3a, iC3b, C4, C4b, C5, and/or C5b. As used herein, “no binding affinity” refers to no detectable binding affinity relative to background with one or more binding affinity assays known in the art, such as, but not limited to, an ELISA assay.

In certain embodiments, the antigen binding proteins are capable of binding an epitope on complement C3, wherein such binding prevents the formation of C3 convertase. In certain embodiments, the antigen binding proteins of the disclosure inhibit the activity of C3 convertase. In certain embodiments, the antigen binding proteins of the disclosure inhibit the C3 convertase amplification loop.

In certain embodiments, the anti-C3 antibodies of the disclosure are expected to have better efficacy and safety in treating GA or other ocular disorders compared to other therapies due to the following properties recited below.

The anti-C3 antibodies of the disclosure may include, but are not limited to, scFv and VHH antibody fragments with a molecular weight of less than about 60 kDa. For example, but in no way limiting, an scFv of the disclosure may have a molecular weight of about 25 kDa and a VHH of the disclosure may have a molecular weight of about 15 kDa, whereas other therapeutic agents may have a larger molecular weight. Based on hydrodynamic radius estimation, the anti-C3 antibodies of the disclosure are expected to have better inhibition for choroidal C3 because they may penetrate the Bruch's membrane more efficiently and enter the choroid of the eye more efficiently.

The anti-C3 antibodies of the disclosure may have a therapeutically effective duration longer than 1 month, which may be a longer duration compared to other therapeutic agents. The increased therapeutically effective duration may be due to the molar concentration of the anti-C3 antibodies of the disclosure, that can reach as high as 7 mM.

The anti-C3 antibodies of the disclosure may be easily injected to the eye compared with other therapeutic agents. The anti-C3 antibodies of the disclosure do not contain PEG, thereby reducing their viscosity. Thus, the viscosity of the anti-C3 antibodies of the disclosure are expected to be lower than that of other therapeutic agents. Reduced viscosity solutions, such as solutions that are less than or equal to 20 centipoise (cP) are more easily injected into the eye because of a reduced back-pressure.

### Expression of Antigen Binding Polypeptides

In one aspect, polynucleotides encoding the binding polypeptides (e.g., antigen binding proteins) disclosed herein are provided. Methods of making a binding polypeptide comprising expressing these polynucleotides are also provided.

Polynucleotides encoding the binding polypeptides disclosed herein are typically inserted in an expression vector for introduction into host cells that may be used to produce the desired quantity of the claimed antibodies, or fragments thereof. Accordingly, in certain aspects, the invention provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding polypeptides may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and/or heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of exogenous DNA into a recipient host cell that changes the genotype and consequently results in a change in the recipient cell. The genetically-modified recipient cell can contain the exogenous sequences by transient or stable transformation. For example, the exogenous sequences can be stably integrated into a genomic sequence of the recipient cell, at a targeted site or in a random site. Cells modified by gene editing methods (e.g., methods using a homologous recombination, transposon-mediated system, loxP-Cre system, CRISPR/Cas9 or TALEN) are within the scope of the present disclosure. In certain embodiments, a stable cell line is generated for production of the antigen binding protein or fragment thereof. This advantageously results in consistent production antigen binding proteins or fragment thereof of uniform quality and yield.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antigen binding proteins featured in the invention can also be expressed in non-mammalian cells such as bacteria or yeast or insect or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus; and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

Thus, in one aspect, a method of manufacturing an antigen binding protein or fragment thereof as described above is provided, comprising the steps of:
i) cultivating a host cell under conditions allowing expression of the protein described herein; and
ii) recovering the protein; and optionally
iii) further purifying and/or modifying and/or formulating the protein.

### Methods of Administering Antigen Binding Proteins

Methods of preparing and administering antigen binding proteins (e.g., antigen binding proteins disclosed herein) to a subject are well known to or are readily determined by those skilled in the art. The route of administration of the antigen binding proteins of the current disclosure may be oral, parenteral, by inhalation, topical, or intraocular. The term parenteral as used herein includes intravenous, intraarterial, intraperitoneal, intramuscular, subcutaneous, rectal or vaginal administration. The term intraocular as used herein includes, but is not limited to, subconjunctival, intravitreal, retrobulbar, or intracameral. The term topical as used herein includes, but is not limited to, administration with liquid or solution eye drops, emulsions (e.g., oil-in-water emulsions), suspensions, and ointments.

In certain embodiments, the antigen binding proteins of the disclosure are administered intraocularly. Delivery of therapeutic compounds to the different structures of the eye, such as the retina, is challenging. The challenges include, but are not limited to, several restrictive ocular barriers, tear mechanisms, including blinking and washing out of delivered compounds, limited local injection volumes, limited local bioavailability, and low tolerance to impurities and contaminants (see, e.g., Patel et al. World J Pharmacol. 2013; 2 (2): 47-64; Morrison et al. Ther. Deliv. 2014; 5 (12): 1297-1315). The antigen binding proteins of the disclosure may overcome these challenges. The antigen binding proteins of the disclosure have a molecular weight of about 60 kDa or less. Examples of antigen binding proteins of about 60 kDa or less include, but are not limited to, scFv, VHH, and Fab fragments. The smaller size of the antigen binding proteins of the disclosure relative to full-length antibodies enables delivery of more therapeutic antibody per an injection. This allows for high concentrations of the antibodies to the eye. The smaller size of the antigen binding proteins of the disclosure may also improve their penetration into the disease-relevant tissues, i.e., the choroid region of the eye. The antigen binding proteins are capable of penetrating one or more layers of the choroid region, including Haller's layer, Sattler's layer, the Choriocapillaris, and Bruch's membrane, thereby targeting complement C3 and C3b within those layers of the choroid region.

In certain embodiments, intraocular administration is achieved with a drug delivery device, such as a suprachoroidal drug delivery device or a subretinal drug delivery device. Suprachoroidal administration procedures involve administration of a drug to the suprachoroidal space of the eye, and are normally performed using a suprachoroidal drug delivery device such as a microinjector with a microneedle (see, e.g., Hariprasad, Retinal Physician; 2016; 13:20-23; Goldstein, 2014, Retina Today 9 (5): 82-87; each of which is incorporated by reference herein in its entirety). The suprachoroidal drug delivery devices that can be used to deposit the antigen binding proteins of the disclosure in the suprachoroidal space include, but are not limited to, suprachoroidal drug delivery devices manufactured by Clearside® Biomedical, Inc. (see, for example. Hariprasad, 2016, supra). The subretinal drug delivery devices that can be used to deposit the antigen binding proteins of the disclosure in the subretinal space via the suprachoroidal space include, but are not limited to, subretinal drug delivery devices manufactured by Janssen Pharmaceuticals, Inc. (see, for example, International Patent Application Publication No. WO 2016/040635).

In certain embodiments intraocular administration is achieved via an intravitreal route. Intravitreal administration is often performed with a syringe and a 27-gauge to 30-gauge needle (see, e.g., Jiang et al. supra).

While all these forms of administration are clearly contemplated as being within the scope of the current disclosure, a form for administration would be a solution for injection, in particular for intravitreal injection. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

In certain embodiments, the antigen binding proteins of the disclosure are formulated in a solution with low viscosity. The viscosity of a solution is measured in units of centipoise (cP). High viscosity antibody solutions may pose challenges for administration of the antigen binding proteins of the disclosure to the eye. For example, solutions with a viscosity above 50 cP may be difficult to administer with a fine needle due to high back-pressure. It is therefore desirable to formulate the antigen binding proteins of the disclosure in a low viscosity solution. In certain embodiments, the antigen binding proteins of the disclosure and pharmaceutical compositions thereof have a viscosity between about 1 cP to about 50 cP. In certain embodiments, the antigen binding proteins of the disclosure and pharmaceutical compositions thereof have a viscosity of less than or equal to about 20 cP, about 15 cP, about 10 cP, about 5 cP, about 4 cP, about 3 cP, about 2 cP, or about 1 cP. Further details regarding antibody viscosity are described in Tomar et al. MAbs. 2016; 8 (2): 216-228 and Fennell et al. MAbs. 2013; 5 (6): 882-895.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05M phosphate buffer, or 0.8% saline. Other common parenteral vehicles include sodium phosphate solutions, Ringer's dextrose, dextrose and sodium chloride, lactated Ringer's, fixed oils and the like. Intravenous vehicles include, but are not limited to, fluid and nutrient replenishers, electrolyte replenishers, such as those based on Ringer's dextrose, and the like. Preservatives and other additives may also be present such as for example, antimicrobials, antioxidants, chelating agents, inert gases and the like. In certain embodiments, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. Isotonic agents, for example, sugars, polyalcohols, or sodium chloride may also be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an antigen binding protein or fragment thereof) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins of the present disclosure, immunoreactive fragments or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the modified antigen binding proteins, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The biological activity of the pharmaceutical compositions defined herein can be determined for instance by complement inhibition assays, for example, but in no way limiting, enzyme immunoassays for the determination of functional classical, lectin, and alternative complement pathway activity in human serum. In certain embodiments, the inhibitory activity of the pharmaceutical compositions defined herein may be evaluated using the Complement system Screen Wieslab® (Euro Diagnostica AB, Malmö, Sweden).

Functional assays to study the ability of the antibodies of the disclosure to inhibit complement pathways can be performed using purified complement components, from which enzymatic complexes are reconstituted on the surface of erythrocytes or artificial matrices, as described by Okroj et al. PLOS One.; 2012; 7 (10): e47245.

The standard 50% hemolytic complement (CH50) assay is also a commonly used method of assessing the ability of compounds to inhibit functional activity of the classical complement pathway, as described in Jaskowski et al. Clinical and Diagnostic Laboratory Immunology; 1999; 6 (1): 137-9.

In certain embodiments, the activity of the CP, LP, and AP complement pathways may be determined by measuring the level of erythrocyte hemolysis in the presence of the antigen binding protein of the disclosure compared to the level of erythrocyte hemolysis in the absence of the antigen binding protein of the disclosure. In certain embodiments, antibody-sensitized sheep erythrocytes may be used to measure complement-dependent hemolysis mediated by the classical pathway. In certain embodiments, antibody-sensitized rabbit erythrocytes may be used to measure complement-dependent hemolysis mediated by the alternative pathway, as described in Tomlinson et al. J Immunol. 1997; 159 (11): 5606-5609.

In certain embodiments, the activity of the CP, LP, and AP complement pathways may be determined by measuring Membrane Attack Complex (MAC) formation in the presence of the antigen binding protein of the disclosure compared to MAC formation in the absence of antigen binding protein of the disclosure. The MAC assay for IgM-mediated activation of the classical complement pathway in human serum leads to deposition of the MAC on IgM coated ELISA plates. MAC formation may be detected with an alkaline phosphatise-labelled antibody to C5b-9. In the presence of the antigen binding protein of the disclosure, the ELISA signal is reduced in a dose dependent manner. For testing the alternative pathway, the MAC assay for LPS-mediated activation of the alternative complement pathway in human serum may be used for deposition of the MAC on LPS coated ELISA plates. An appropriate MAC assay includes, but is not limited to, the Pacific Biomarkers Complement Membrane Attack Complex (SC5b-9) ELISA assay.

“Efficacy” or “in vivo efficacy” as used herein refers to the response to a therapy by the pharmaceutical composition of the disclosure, using e.g., standardized response criteria, such as standard ophthalmological response criteria. The success or in vivo efficacy of the therapy using a pharmaceutical composition of the disclosure refers to the effectiveness of the composition for its intended purpose, i.e., the ability of the composition to cause its desired effect, i.e., inhibition of the complement pathway in the eye. The in vivo efficacy may be monitored by established standard methods for the various ocular diseases. Methods of monitoring include, but are not limited to, the Amsler grid test, opthtalmoscopy, ocular fundus microscopy, ocular computer tomography, and optical coherence tomography. In addition, various disease specific clinical chemistry parameters and other established standard methods may be used.

### Antibody Engineering and Optimization

The antigen binding proteins of the disclosure may be engineered or optimized. As used herein, “optimized” or “optimization” refers to the alteration of an antigen binding protein to improve one or more functional properties. Alteration includes, but is not limited to, deletions, substitutions, additions, and/or modifications of one or more amino acids within an antigen binding protein.

As used herein, the term “functional property” is a property of an antigen binding protein for which an improvement (e.g., relative to a conventional antigen binding protein) is desirable and/or advantageous to one of skill in the art, e.g., in order to improve the manufacturing properties or therapeutic efficacy of an antigen binding protein. In one embodiment, the functional property is stability (e.g., thermal stability). In another embodiment, the functional property is solubility (e.g., under cellular conditions). In yet another embodiment, the functional property is aggregation behavior. In still another embodiment, the functional property is protein expression (e.g., in a prokaryotic cell). In yet another embodiment the functional property is refolding behavior following inclusion body solubilization in a manufacturing process. In certain embodiments, the functional property is not an improvement in antigen binding affinity. In another embodiment, the improvement of one or more functional properties has no substantial effect on the binding affinity of the antigen binding protein.

In certain embodiments, the antigen binding protein of the disclosure is an scFv and is optimized by identifying preferred amino acid residues to be substituted, deleted, and/or added at amino acid positions of interest (e.g., amino acid positions identified by comparing a database of scFv sequences having at least one desirable property, e.g., as selected with Quality Control (QC) assay, versus a database of mature antibody sequences, e.g., the Kabat database) in an antigen binding protein. Thus, the disclosure further provides “enrichment/exclusion” methods for selecting a particular amino acid residue. Still further, the disclosure provides methods of engineering antigen binding proteins (e.g., scFvs) by mutating particular framework amino acid positions identified using the “functional consensus” approach described herein. In certain embodiments, the framework amino acid positions are mutated by substituting the existing amino acid residue by a residue which is found to be an “enriched” residue using the “enrichment/exclusion” analysis methods described herein. In one aspect, the disclosure provides a method of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database. ScFV optimization is described in further detail in WO2008110348, WO2009000099, WO2009000098, and WO2009155725, all of which are incorporated herein by reference.

### Humanization:

In certain embodiments, the antigen binding proteins of the disclosure may be humanized. As used herein, the term “humanized” refers to a non-human donor antibody that has been modified to increase their similarity to antibodies produced naturally in humans. As used herein, the term “humanization” refers to the process of humanizing a non-human donor antibody. Humanization may be achieved by grafting CDRs of non-human donor antibodies (e.g., rabbit or llama antibody CDRs) onto human or humanized antibody acceptor framework regions, such as soluble and stable light chain and/or heavy chain human antibody framework regions. A general method for grafting CDRs into human acceptor frameworks has been disclosed by Winter in U.S. Pat. No. 5,225,539 and by Queen et al. in WO199007861, which are hereby incorporated by reference. Appropriate acceptor framework regions may exhibit superior functional properties, such as improved solubility and stability. In certain embodiments, the antigen binding proteins of the disclosure are rabbit antibodies. The CDRs of said rabbit antibodies may be grafted into a universal acceptor framework region, such as the framework regions described in WO2009155726, incorporated herein by reference.

In certain embodiments, the human frameworks for the humanization/stabilization of non-human antibodies or the stabilization of human antibodies relates to the replacement of a κ joining segment in a κ variable light domain by a λ joining segment resulting in a ζ-λ chimeric variable light domain with improved protein stability and reduced aggregation propensity. It further relates to the mutation of the k consensus residue at position AHo101 and replacement by a λ consensus residue to support packing of the λ joining segment in a κ-λ chimeric variable light domain to further improve protein stability and to further reduce aggregation propensity. Further details regarding these human framework regions are described in WO2014206561 and WO2019057787, incorporated herein by reference.

### Methods of Treating Complement C3-Mediated Diseases and Disorders

Methods of treating complement C3-mediated diseases and disorders using the antigen binding proteins described herein in a subject suffering from a complement C3-mediated disease or disorder are provided.

In certain embodiments, the complement C3-mediated disease or disorder is selected from a group consisting of age-related macular degeneration (AMD), geographic atrophy (GA), neovascular glaucoma, diabetic retinopathy, retinopathy of prematurity, retrolental fibroplasia, autoimmune uveitis, chorioretinitis, retinitis, rheumatoid arthritis, psoriasis and atherosclerosis. In certain embodiments, the C3-mediated disease is a form of AMD. AMD is generally divided into two main classes, dry AMD and wet AMD. Dry AMD, also known as nonexudative AMD, is characterized by the presence of drusen (yellow deposits) in the macular region. Wet AMD, also known as exudative AMD or neovascular AMD, is characterized by the growth of abnormal blood vessels from the choroid underneath the macula. This process is also called choroidal neovascularization and the new blood vessels may leak fluid, such as blood, into and around the retina. Geographic Atrophy, also known as atrophic AMD or advanced dry AMD, is an advanced form of AMD that may result in the progressive and irreversible loss of retinal cells.

It is particularly challenging to treat ocular disorders, such as AMD described above. As recited previously, delivery of therapeutic agents to the eye is limited due to several barriers, including, but not limited to, blood-retinal-barriers, such as the RPE. The ability to penetrate the RPE and enter the choroid of the eye would enhance the therapeutic potential of drugs. In certain embodiments, the antigen binding proteins of the disclosure are capable of penetrating the RPE and Bruch's membrane of the choroid region of the eye, thereby targeting complement C3 in the choroid region. The ability of the antigen binding proteins of the disclosure to penetrate the RPE and Bruch's membrane improves their therapeutic potential in the treatment of complement C3-mediated diseases or disorders. The antigen binding proteins of the disclosure are capable of penetrating the RPE and Bruch's membrane in part due to their size, which is sufficiently low to facilitate penetration. In certain embodiments, the size of the antigen binding proteins of the disclosure are measured by molecular weight. In certain embodiments, the antigen binding proteins of the disclosure have a molecular weight that is less than about 60 kDa. In certain embodiments, the antigen binding proteins of the disclosure are about 20 kDa to about 30 kDa or about 10 kDa to about 20 kDa. In certain embodiments, the antigen binding proteins of the disclosure are about 25 kDa. In certain embodiments, the antigen binding proteins of the disclosure are about 15 kDa. In certain embodiments, the size of the antigen binding proteins of the disclosure are measured by their hydrodynamic radius. In certain embodiments, the antigen binding proteins of the disclosure have a hydrodynamic radius of less than or equal to about 3.0 nm. In certain embodiments, the antigen binding proteins of the disclosure have a hydrodynamic radius of less than or equal to about 2.5 nm. In certain embodiments, the antigen binding proteins of the disclosure have a hydrodynamic radius of less than or equal to about 2.0 nm.

In one aspect, the disclosure provides a method of inhibiting the activity of the complement Classical pathway (CP), Lectin pathway (LP), and Alternative pathway (AP), the method comprising contacting complement C3 with an antigen binding protein or fragment thereof which binds an epitope on complement C3. The ability of the antigen binding proteins of the disclosure to inhibit all three complement pathways further improves their therapeutic potential in the treatment of complement C3-mediated diseases or disorders. Without wishing to be bound by theory, inhibiting all three complement pathways may improve the therapeutic potential of the antigen binding proteins of the disclosure by preventing the disease-promoting effects of one active pathway from compensating for the other inactivated pathways.

In certain embodiments, the antigen binding protein or fragment thereof is capable of approximately equivalent inhibition of the activity of the CP, LP, and AP complement pathways. For example, but in no way limiting, the antigen binding protein or fragment thereof is capable of inhibiting the activity of the CP pathway by at least 80%, capable of inhibiting the activity of the LP by at least 80%, and capable of inhibiting the activity of the AP by at least 80%. In certain embodiments, the inhibition of the activity of the CP, LP, and AP complement pathways is at least about 80%, at least about 85%, at least about 90%, or at least about 95%.

In another aspect, the disclosure provides a method of inhibiting the activity of choroidal-localized complement C3 through the intraocular administration of an antigen binding protein or fragment thereof which binds an epitope on complement C3. The activated complement pathway in the choroid region of the eye may contribute to complement C3-mediated diseases or disorders. It is therefore an object of the disclosure to provide antigen binding proteins that are capable of penetrating or diffusing into the choroid region and targeting complement C3 and C3b. In certain embodiments, the antigen binding proteins of the disclosure inhibit the activity of C3 convertase in the choroid region of the eye. In certain embodiments, the antigen binding proteins of the disclosure inhibit the C3 convertase amplification loop in the choroid region of the eye.

### Medical Use

The invention also relates to an antigen binding protein or fragment thereof as disclosed herein for use in a method of treating a complement C3-mediated disease or disorder in a subject. All the technical features described in the present disclosure regarding the antigen binding proteins or fragments thereof are applicable.

### Kits

The invention also encompasses kits comprising at least one antigen binding protein or fragment thereof as described herein. In one embodiment, the kit includes a composition containing an effective amount of said antigen binding protein or fragment thereof in unit dosage form. Such kit may comprise a sterile container comprising the composition; non-limiting examples of such containers include, without being limited to, vials, ampoules, bottles, tubes, syringes, blister-packs. In some embodiments, the composition is a pharmaceutical composition and the containers is made of a material suitable for holding medicaments. In one embodiment, the kit may comprise in a first container the antigen binding protein or fragment thereof in lyophilized form and a second container with a diluent (e.g., sterile water) for reconstitution or dilution of the antigen binding protein of fragment thereof. In some embodiments, said diluent is a pharmaceutically acceptable diluent.

Typically, the kit will further comprise a separate sheet, pamphlet or card supplied in or with the container with instructions for use. If the kit is intended for pharmaceutical use, it may further comprise one or more of the following: information for administering the composition to a subject having a complement C3-mediated disease or disorder and a dosage schedule, description of the therapeutic agent, precautions, warnings, indications, counter-indications, overdosage information and/or adverse reactions.

Diagnostic Applications and/or Detection

The antigen binding protein or fragment thereof of the instant invention may be used for detection or diagnostic purposes in vivo and/or in vitro. For example, a wide range of immunoassays involving antigen binding proteins for detecting the expression in specific cells or tissues are known to the skilled person. For such applications, the antigen binding protein or fragment thereof disclosed herein may be either labeled or unlabeled. For example, but in no way limiting, an unlabeled antigen binding protein may be used and detected by a secondary antibody recognizing an epitope on the antigen binding protein described herein. In another embodiment the antigen binding protein or fragment thereof is conjugated with one or more substances which can be recognized by a detector substance(s), e.g., the antigen binding protein or fragment thereof being conjugated with biotin which can be detected by streptavidin. In certain embodiments, the antigen binding protein or fragment thereof is useful for detecting the presence of C3 and/or C3b in a sample. In certain embodiments, the sample is a biological sample. As used herein, the term “detecting” encompasses quantitative and/or qualitative detection. In certain embodiments, a biological sample comprises a cell or tissue, such as retinal tissue, from a human patient.

In certain embodiments, the method comprises contacting the biological sample with at least one antigen binding protein or fragment thereof of the instant invention; permitting formation of complexes between C3 (if present) and the antigen binding protein or fragment thereof in the sample; and then, detecting said antigen binding protein or fragment thereof. In preferred embodiments, the antigen binding protein or fragment thereof is capable of binding both complement C3 and C3b.

In one embodiment, the antigen binding protein or fragment thereof is detected by a detectable signal. In another embodiment, the antigen binding protein or fragment thereof is detected by ELISA, immunocytochemistry (ICC), immunohistochemistry (IHC), Western Blotting and/or Flow cytometry.

The biological sample may be a tissue sample, such as a retinal tissue. The tissue sample may be a fixed tissue sample, such as a formalin-fixed and paraffin-embedded tissue sample.

In one embodiment, such method is used for selecting patients, i.e., to determine a subject's eligibility for therapy with the antigen binding proteins or fragments thereof as described herein.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Generation and Characterization of Anti-C3 Antibody Library

To generate antibodies that inhibit the complement cascade more efficiently than is possible with partial inhibitors of complement, it was hypothesized that a broad collection of anti-C3 antibodies with diverse epitope recognition increases the probability for isolation of antibodies with the desired function. For this, large antibody phage libraries were constructed using the genomic information coding for antibody variable domains derived from B cells of animals immunized with C3.

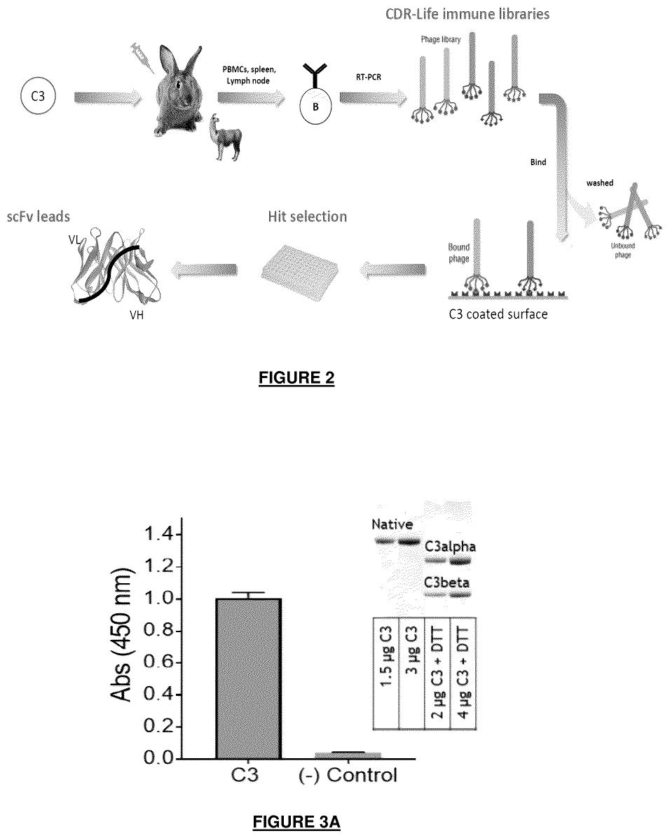

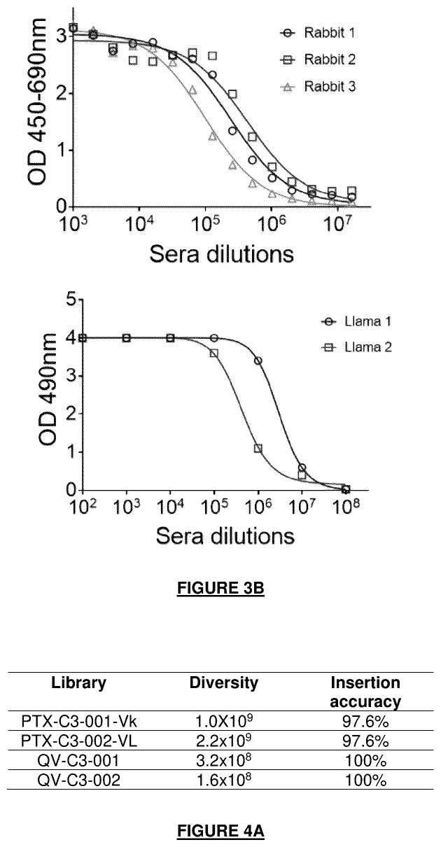

To generate numerous antibodies able to recognize different epitopes on C3, 3 New Zealand white rabbits and 2 llamas were immunized with native human C3 protein purified from serum (FIG. 2). Each animal received 4 injections of the C3 protein at different timepoints with complete or incomplete Freund's adjuvant (FIG. 3A). The immune response of each animal was tested with an ELISA to quantify anti-C3 antibodies present in serum samples of the immunized animals. Antibody titers in sera indicated excellent immune responses (FIG. 3B).

scFv antibody cDNA libraries were constructed from the RNA extracted from isolated PBMCs and spleen lymphocytes from rabbits via PCR amplification. Coding sequences for the variable light- and heavy-domain were amplified separately and linked through a series of overlap polymerase chain reaction (PCR) steps to give the final scFv products.

For the llamas, a large bleed was performed from which RNA was isolated and transcribed into cDNA using a reverse transcriptase Kit. The cDNA was cleaned the heavy chain fragments were amplified using primers annealing at the leader sequence region and at the CH2 region.

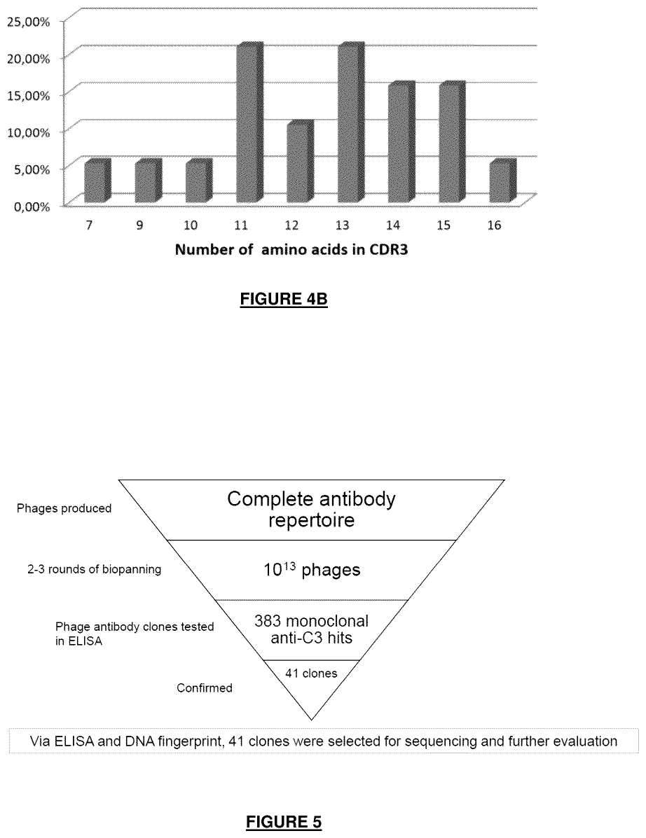

The amplified DNA sequences coding for the scFvs from rabbits and VHHs from llamas were digested using appropriate restriction enzymes and were subsequently ligated into the phagemid vectors. The phagemid vectors were transformed into TG1 electrocompetent cells which are well suited for antibody phage display library creation. These processes resulted in four antibody libraries with sizes of greater than 108 clones and were close to 100% insert percentage (FIG. 4A and FIG. 4B).

### Example 2—Screen of Anti-C3 Antibody which Inhibits all Three Complement Pathways

C3 is a large protein composed of 13 different domains and a molecular size of 185 kilodaltons. During complement activation, C3 undergoes proteolytic cleavage and structural modifications at different sites. The C3 derived fragments exert different effector functions and form convertases that fuel amplification loops of the complement pathways. The enzyme C3-convertase has the ability to cleave multiple C3 molecules into C3b to generate more C3 convertase in a powerful amplification loop, resulting in the full activation of the complement system. The screen described herein was used to identify antibodies that bind different epitopes on both C3 and C3b and effectively block all three pathways of complement activation (classical, lectin, and alternative).

To screen for anti-C3 antibody with high affinities, scFvs and VHH antibodies displayed on phages were produced and submitted to several rounds of biopanning (selection) against native human C3 purified from serum. Stringency of the selection was increased with each round by either decreasing the concentration of C3 protein used in the biopanning or increasing the stringency of the washes. Approximately 380 monoclonal phages were selected and screened for their ability to bind C3 in ELISA assays (FIG. 5).

Based on ELISA data and DNA fingerprint, 41 phage clones were selected for sequencing and produced recombinantly as antibody proteins and evaluated for their ability to bind human C3 and C3b and for further characterization (FIG. 5).

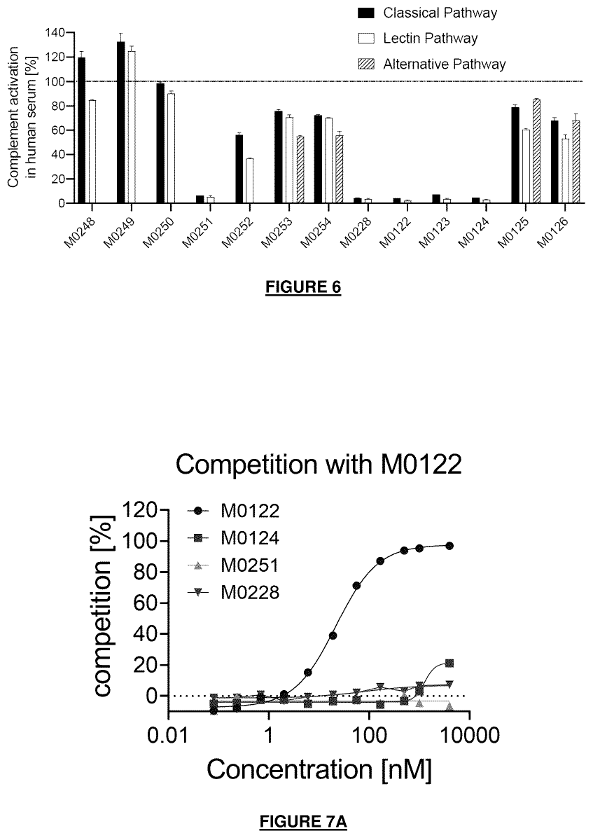

To identify antibodies that block all 3 complement pathways, antibodies were screened using an enzyme immunoassay for the qualitative determination of functional classical, lectin and alternative complement pathways in human serum using the Wieslab® Complement system Screen (Svar Life Science AB, Malmö, Sweden). The amount of C5b-C9 neoantigen generated is proportional to the functional activity of complement pathways. As shown in FIG. 6, five antibodies, M0251, M0228, M0122, M0123 and M0124, were capable of inhibiting all three complement pathways at fixed concentrations of 2 μM by at least 90% in human serum (Quidel).

### Example 3—Characterization of Anti-C3 Antibodies: M0251, M0228, M0122, M0123, and M0124

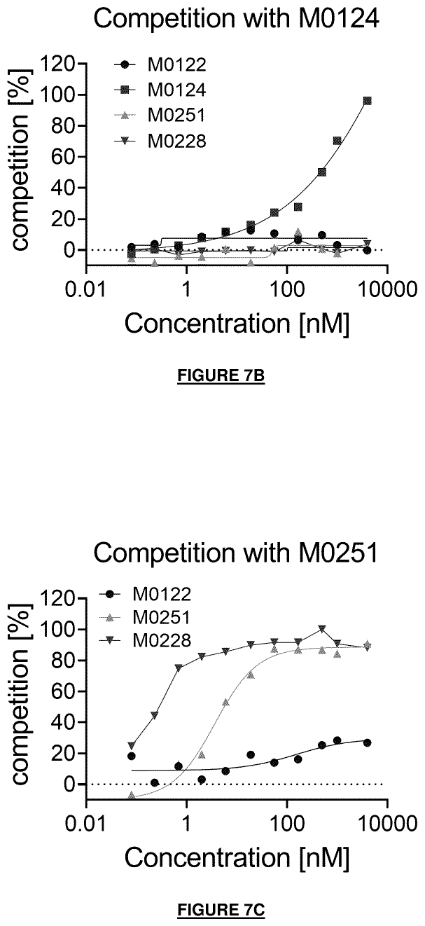

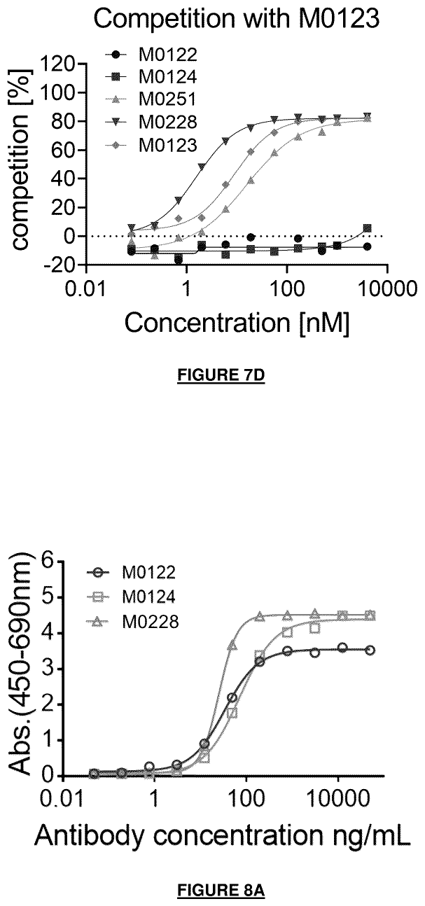

M0251, M0228, M0122, M0123 and M0124 were tested in a pairwise combinatorial manner to identify those that target the same region (epitope) on C3. Briefly, one antibody was labelled via biotinylation and incubated together with other antibody clones in a C3 binding ELISA. Those anti-C3 antibodies that competed for the same binding region were considered to share similar epitopes and therefore to have similar functions. This information enables to reduce the number of potential antibody candidates while maintaining epitope diversity. Out of the five antibodies inhibiting all three complement pathways, M0251, M0228 and M0123 were considered to share the same epitope on C3 (FIG. 7D). Inhibiting leads were assumed to bind three different epitopes on C3 (FIG. 7A-7D).

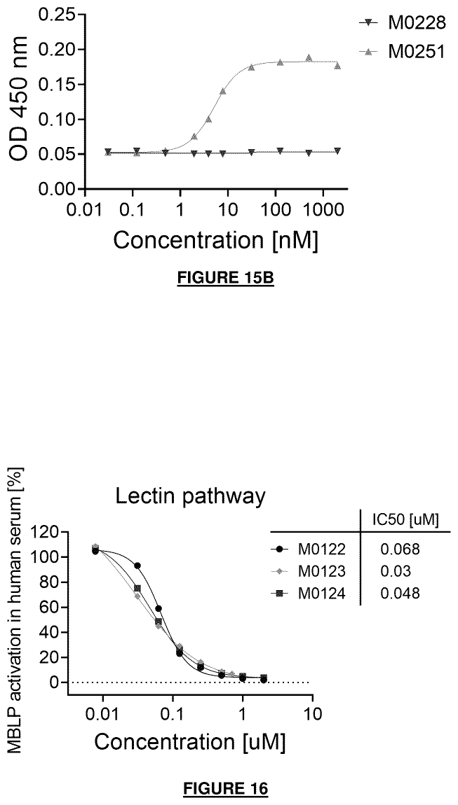

Antibodies identified as capable of inhibiting all three complement pathways were assessed for their ability to bind cynomolgus monkey C3 in ELISA. Briefly, 96 well ELISA plates were coated with polyclonal goat antiserum deemed to be cross-reactive to cyno C3 and followed by a secondary association with a custom preparation of cynomolgus monkey serum (BioIVT, NB-151558). Serial dilutions of the antibody molecules were added to the ELISA plate and antibodies binding to cyno C3 were detected with a rabbit anti-human Kappa HRP antibody (Abcam, ab202549) or a mouse anti-His Tag HRP antibody (R&D Systems, MAB050H). Leads M0122, M0124 and M0251 show binding in a dose response manner to cynomolgus monkey C3. Interestingly, although M0251, M0228 and M0123 compete for the same epitope on human C3, only M0251 showed binding activity for cyno C3 (FIGS. 15A and 15B).

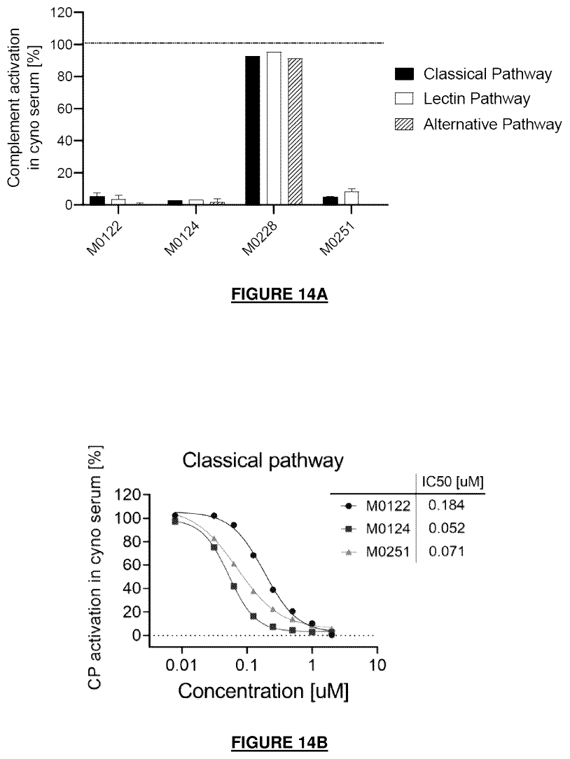

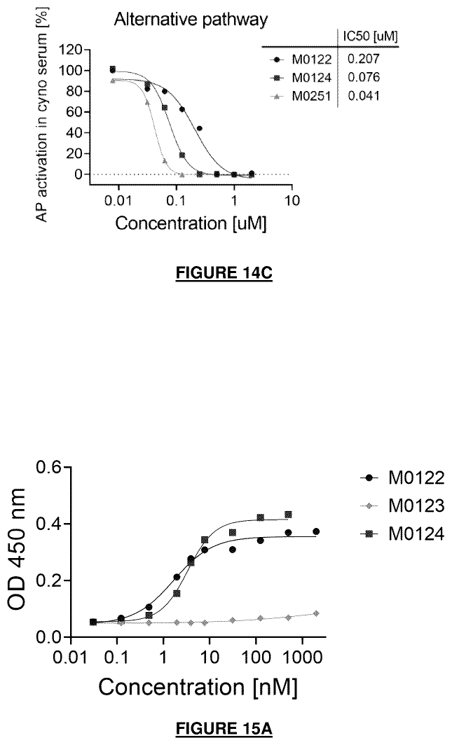

The Wieslab Complement system Screen was used to assess the ability of anti-C3 antibodies to inhibit all pathways of complement activation in cynomolgus monkey serum. Anti-C3 antibodies were added to the custom preparation of cyno serum. FIG. 14A shows potent inhibition of all three complement pathways by M0122, M0124 and M0251 at fixed concentrations of 2 μM, suggesting that M0122, M0124 and M0251 are potent inhibitors of complement mediated MAC formation in cyno serum. M0228 showed no inhibitory activity of the complement pathway in cyno serum, confirming the lack of binding activity observed for this antibody to cyno C3 (FIG. 14B). Dose dependent inhibition of the classical and alternative pathway in cynomolgus monkey serum was further assessed for M0122, M0124 and M0251 using the corresponding Wieslab complement system kits (FIGS. 14B and 14C).

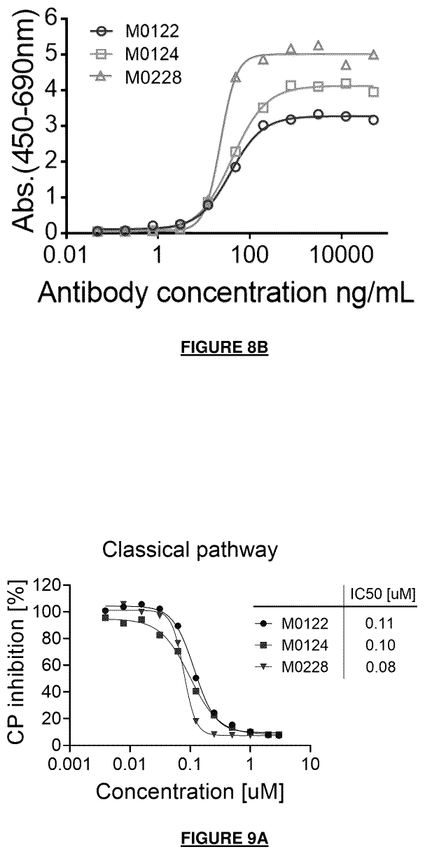

M0122, M0124 and M0228 were evaluated for their ability to bind both human C3 and C3b in a direct binding ELISA assay (FIG. 8A and FIG. 8B). Briefly, 96 well ELISA plates were coated with purified native human C3 or C3b (Complement Technology, A113 and A114). Serial dilutions of antibody molecules were added to the plate and detected by a rabbit anti-human Kappa HRP antibody (Abcam, ab202549) or a rabbit anti-His Tag HRP antibody (Abcam, ab1187).M0122, M0124 and M0228 show high affinity binding to both human C3 and C3b. The binding kinetics of M0122, M0124 and M0228 for human C3 were further analyzed by Biolayer interferometry exhibiting affinities in the low picomolar range (FIG. 10).

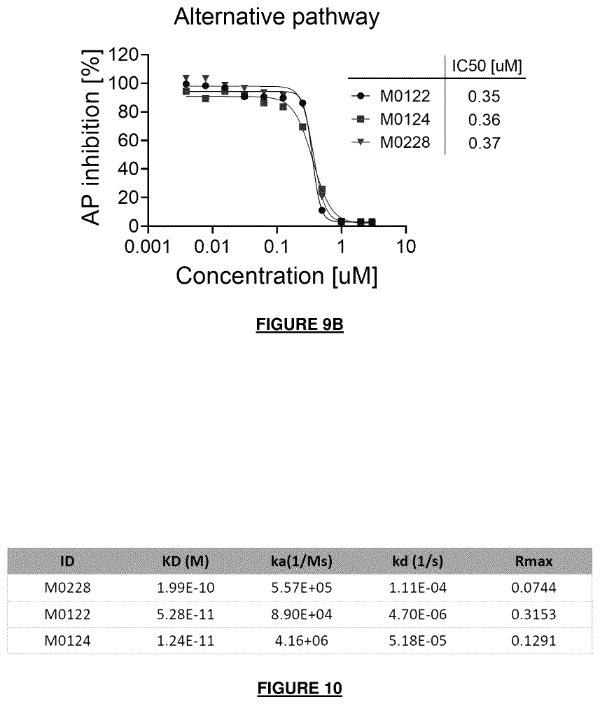

Dose dependent inhibition of the alternative and classical pathway in human serum was assessed for M0122, M0124 and M0228 using the corresponding Wieslab complement systems kits Anti-C3 antibodies M0122, M0124 and M0228 show strong inhibition of the alternative and classical pathway in human serum (FIG. 9A and FIG. 9B). Anti-C3 antibodies M0122, M0123 and M0124 were further evaluated for their ability to inhibit the lectin pathway in dose response manner. FIG. 16 shows efficient inhibition of the lectin pathway in human serum. All combined, these results further support efficient inhibition of all three pathways of complement activation by the antibodies of the invention.

### Example 4—the Anti-C3 Antibodies are More Likely to Penetrate Bruch's Membrane than APL-2

It is now known that the complement system plays a role in the pathogenesis of geographic atrophy. However, is not yet fully understood how complement activity is compartmentalized in the eye and whether efficacy of a GA therapy depends upon delivering the therapeutics to the correct anatomical sites within the eye. We hypothesize that better penetration into disease-relevant retinal tissues (i.e. RPE, Bruch's membrane and choroid) may be necessary to achieve greater reduction of lesion growth in GA. The inner part of choroid is called choriocapillaris, which contains capillaries that are separated by a sheet of extracellular membrane called Bruch's membrane (BrM) (FIG. 11).

The Bruch's membrane is selectively permeable to antibodies and biologics. As reported by Clark et al. (Front. Immunol. 2017. 8:1-10), complement pathway proteins cannot pass the Bruch's membrane except FHL-1, factor D and C5a. Overall, antibodies and biologics with large hydrodynamic radii are less likely to pass Bruch's membrane. As shown in Table 3 below, other than APL-2 and CDR2 (an anti-C3 scFv of the disclosure), the listed molecules with hydrodynamic radii larger than 3.00 cannot pass Bruch's membrane except FHL-1; on the other hand, all the listed molecules with hydrodynamic radii larger than 3.00 can pass Bruch's membrane except C3a.

TABLE 3
Size Factors of A List of Biologies

| | Hydrodynamic | | Diffusion |
|---|---|---|---|
| Protein | radius (nm) | MW (kDa) | (>70 yr old BM) |
| APL-2* | ~7-8 | 43 (linear PEG) | Nk |
| IgG | ~6 | 150 | No |
| Factor H | 5.56 | 155 | No |
| C3 | 4.84 | 180 | No |
| FHL-1 | 4.40 | 49 | Yes |
| Factor B | 3.22 | 83 | No |
| Factor I | 3.07 | 65 | No |
| Fab fragment | 2.91 | 50 | Yes |
| CDR2* | 2.5 | 26 | Nk |
| Factor D | 2.08 | 24 | Yes |
| C3a | 1.56 | 9 | No |
| C5a | 1.63 | 8.3 | Yes |

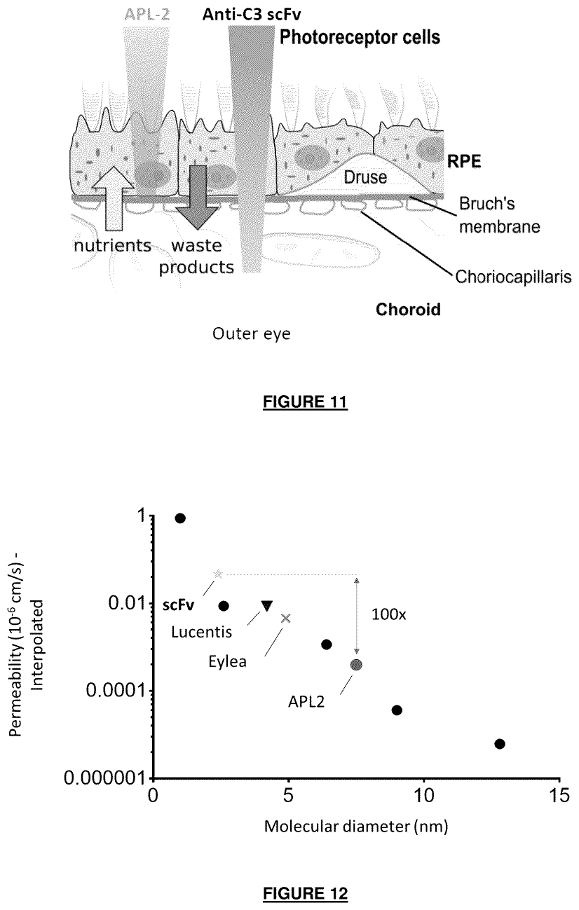

In addition, Pitkänen et al. (Invest Ophthalmol Vis Sci. 2005; 46 (2): 641-6) studied the permeability of fresh RPE-choroid specimens from bovine eyes for carboxyfluorescein, fluorescein isothiocyanate (FITC)-labeled dextrans with molecular masses from 4 to 80 kDa. We took plotted permeability against the molecular sizes (FIG. 12, black dots). We also derived permeability values of scFv, lucentis, eylea and APL2 based on hydrodynamic radii using the study performed by Hirvonen et al. (Pharm Res. 2016; 33 (8): 2025-32), and plotted the permeability against the molecular weights in the same graph (FIG. 12, colored dots). The trend indicates that the larger the molecular weight is, the poorer the permeability for Bruch's membrane is.

We predicted that it is highly likely that the anti-C3 antibodies of the disclosure as antibody fragments, such as, but not limited to, an scFv or VHH format, with hydrodynamic radii of about 2.5 nm and smaller, can better permeate Bruch's membrane than APL-2 (two anti-C3 cyclic APL-1 peptides linked to 40 kDa linear PEG, total of 43 kDa), of which the hydrodynamic radius is at least 7 nm.

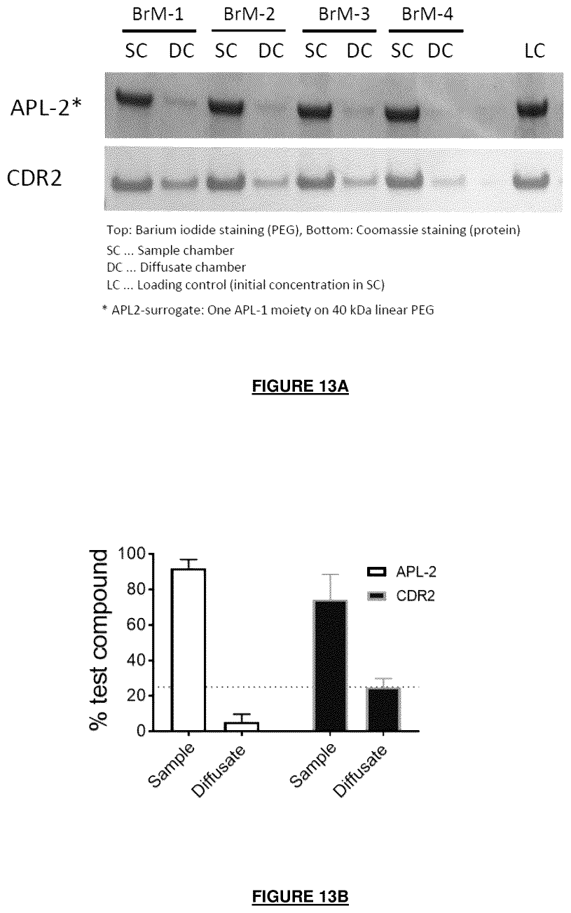

To test this hypothesis, the ability of anti-C3 molecules to cross BrM was evaluated using enriched porcine BrM mounted on Ussing chambers. Briefly, the enriched Bruch's membrane was isolated from porcine eyes and mounted in a Ussing diffusion chamber (Multi Channel Systems MCS GmbH, Cat. No. 660026). Once mounted, the 5-mm diameter Bruch's membrane was the only barrier between two identical compartments. Both sides of the Bruch's membrane were washed with 1 ml of PBS for at least 5 min at room temperature. For a leakage test, 1 ml of PBS was added to the sample chamber and leakage into the second compartment was tracked for 5 min. If no leaks were detected, which would indicate a compromise in membrane integrity, antibody proteins were added to the sample chamber in 1 ml PBS at 100 μg/ml and 1 ml PBS was added to the second compartment (diffusate chamber). The entire Ussing chamber was incubated at room temperature for 24 h with gentle shaking to avoid generating gradients of diffusing proteins. Samples from each chamber (15 μl) were analyzed by gel electrophoresis. Pre-cast 4-12% NuPAGE Bis Tris SDS gels (Thermo Fisher Scientific) were run for 40 min at 200 V under reducing conditions. Gels were either stained with Instant Blue stain (Expedeon) for 60 min at room temperature for detection of antibody proteins or with Barium iodide solution for detection of PEG (fixing of gel with 0.1 M perchloric acid, which after 15 min is replaced with a pre-mix of 20 ml of 5% BaCl₂ and 8 ml 0.1 M iodine solution, which after 10 min is repeatedly replaced with deionized water every 10 min for 1 h). In order to calculate the percentage of protein in the sample or diffusate chambers, band densities in the Instant Blue stained or BaI₂ stained SDS gels was measured using ImageJ software. The average intensity of these bands was compared to the density of control bands that represent 100% loaded protein (i.e., 15 μl of 100 μg/ml). Then the calculated percentage protein was plotted±SD. The ability to cross porcine BrM was compared for a scFv-derivative of M0123 (26 kDa) and a APL-2 surrogate (one anti-C3 cyclic APL-1 peptide linked to 40 kDa linear PEG, total of 42 kDa) that were simultaneously incubated on BrM preparations from four different porcine eyes. Significantly higher amounts of the scFv crossed the BrM in all four membrane preparations compared to the APL-2 surrogate (FIG. 13A and FIG. 13B).

1. An antigen binding protein or fragment thereof which binds an epitope on complement C3, wherein the antigen binding protein or fragment thereof is capable of inhibiting the pathways of complement activation, including the Classical pathway (CP), the Lectin pathway (LP), and the Alternative pathway (AP).

2. The antigen binding protein or fragment thereof of claim 1, wherein said antigen binding protein or fragment thereof (a) binds complement C3 and C3b; (b) binds an epitope on complement C3, wherein such binding prevents the formation of C3 convertase; (c) penetrates Bruch's membrane; and/or (d) competes with one or more antigen binding proteins, including M0122, M0123, M0124, M0228, and M0251.

3-5. (canceled)

6. The antigen binding protein or fragment thereof of claim 1, comprising a single chain variable fragment (scFv), a Fab fragment, a Fab′ fragment, a Fv fragment, a diabody, a small antibody mimetic or a single domain antibody, such as a sdAb, a sdFv, a nanobody, a V-Nar or a VHH.

7. The antigen binding protein or fragment thereof of claim 1, comprising a CDR-H3 having at least 80% identity to a sequence of the group consisting of SEQ ID NO: 3, SEQ ID NO: 6, SEQ ID NO: 9, SEQ ID NO: 15, and SEQ ID NO: 21.

8. The antigen binding protein or fragment thereof of claim 1, comprising a variable heavy chain (VH), and a variable light chain (VL),
wherein the VH comprises a CDR-H1 sequence selected from the group consisting of SEQ ID NO: 1, 4, 7, 13, and 19, a CDR-H2 sequence selected from the group consisting of SEQ ID NO: 2, 5, 8, 14, and 20, a CDR-H3 sequence selected from the group consisting of SEQ ID NO: 3, 6, 9, 15, and 21; and
wherein the VL comprises a CDR-L1 sequence selected from the group consisting of SEQ ID NO: 10, 16, and 22, a CDR-L2 sequence selected from the group consisting of SEQ ID NO: 11, 17, and 23, and a CDR-L3 sequence selected from the group consisting of SEQ ID NO: 12, 18, and 24.

9. The antigen binding protein or fragment thereof of claim 1, wherein the VH has at least 80% identity to a sequence of the group consisting of SEQ ID NO: 25, 26, 27, 29, and 31, and/or the VL has at least 80% identity to a sequence of the group consisting of SEQ ID NO: 28, 30, and 32.

10. The antigen binding protein or fragment thereof of a claim 1, comprising a VH and a VL, wherein the VH comprises a CDR-H1 sequence of SEQ ID NO: 7, a CDR-H2 sequence of SEQ ID NO: 8, and a CDR-H3 sequence of SEQ ID NO: 9; and wherein the VL comprises a CDR-L1 sequence of SEQ ID NO: 10, a CDR-L2 sequence of SEQ ID NO: 11, and a CDR-L3 sequence of SEQ ID NO: 12, optionally wherein the VH comprises the amino acid sequence of SEQ ID NO: 27 and the VL comprises the amino acid sequence of SEQ ID NO: 28.

11. (canceled)

12. The antigen binding protein or fragment thereof of claim 1, comprising a VH and a VL, wherein the VH comprises a CDR-H1 sequence of SEQ ID NO: 13, a CDR-H2 sequence of SEQ ID NO: 14, and a CDR-H3 sequence of SEQ ID NO: 15; and wherein the VL comprises a CDR-L1 sequence of SEQ ID NO: 16, a CDR-L2 sequence of SEQ ID NO: 17, and a CDR-L3 sequence of SEQ ID NO: 18, optionally wherein the VH comprises the amino acid sequence of SEQ ID NO: 29 and the VL comprises the amino acid sequence of SEQ ID NO: 30.

13. (canceled)

14. The antigen binding protein or fragment thereof of claim 1, comprising a VH and a VL, wherein the VH comprises a CDR-H1 sequence of SEQ ID NO: 19, a CDR-H2 sequence of SEQ ID NO: 20, and a CDR-H3 sequence of SEQ ID NO: 21; and wherein the VL comprises a CDR-L1 sequence of SEQ ID NO: 22, a CDR-L2 sequence of SEQ ID NO: 23, and a CDR-L3 sequence of SEQ ID NO: 24, optionally wherein the VH comprises the amino acid sequence of SEQ ID NO: 31 and the VL comprises the amino acid sequence of SEQ ID NO: 32.

15. (canceled)

16. The antigen binding protein or fragment thereof of claim 1, comprising a VHH domain, wherein the VHH domain comprises a CDR-H1 sequence of SEQ ID NO: 1, a CDR-H2 sequence of SEQ ID NO: 2, and a CDR-H3 sequence of SEQ ID NO: 3, optionally wherein the VHH domain comprises the amino acid sequence of SEQ ID NO: 25.

17. (canceled)

18. The antigen binding protein or fragment thereof of claim 1, comprising a VHH domain, wherein the VHH domain comprises a CDR-H1 sequence of SEQ ID NO: 4, a CDR-H2 sequence of SEQ ID NO: 5, and a CDR-H3 sequence of SEQ ID NO: 6, optionally wherein the VHH domain comprises the amino acid sequence of SEQ ID NO: 26.

19. (canceled)

20. The antigen binding protein or fragment thereof of claim 1, comprising a binding affinity for C3 and C3b of at least about 10⁻⁸ M; a binding affinity for C3 and C3b of about 10⁻¹⁰ M to about 10⁻¹² M; approximately equivalent binding affinity for C3 and C3b; wherein the binding affinity for C3 is within a factor of 10 of the binding affinity for C3b; comprising a binding affinity for C3a, iC3b, C4, C4b, C5, and/or C5b of about 10⁻⁴ M or weaker; comprising weaker binding affinity for C3a, iC3b, C4, C4b, C5, and/or C5b compared to the binding affinity for C3 and C3b; or comprising no binding affinity for C3a, iC3b, C4, C4b, C5, and/or C5b.

21-27. (canceled)

28. The antigen binding protein or fragment thereof of claim 1, capable of inhibiting the activity of any one of the group consisting of the CP, LP, and AP complement pathway or capable of approximately equivalent inhibition of the activity of the CP, LP, and AP complement pathway.

29-36. (canceled)

37. The antigen binding protein or fragment thereof of claim 1, comprising a molecular weight of about 60 kDa or less; a molecular weight of about 20 kDa to about 30 kDa; a molecular weight of about 10 kDa to about 20 kDa; a molecular weight of about 25 kDa; or a molecular weight of about 15 kDa; or wherein the antigen binding protein or fragment thereof comprises cross-reactivity with cynomolgus C3.

38-42. (canceled)

43. A pharmaceutical composition comprising the antigen binding protein or fragment thereof of claim 1, and a pharmaceutically acceptable carrier, optionally comprising low viscosity; optionally wherein the viscosity is between about 1 cP to about 50 cP, or less than or equal to about 20 cP.

44-46. (canceled)

47. An isolated nucleic acid molecule encoding the antigen binding protein or fragment thereof of claim 1, or an expression vector comprising said nucleic acid molecule, or a host cell comprising said expression vector.

48-49. (canceled)

50. A method of manufacturing the antigen binding protein or fragment thereof of claim 1 comprising the steps of:
(i) cultivating the host cell comprising an expression vector comprising an isolated nucleic acid molecule encoding said antigen binding protein or fragment under conditions allowing expression of the protein;
(ii) recovering the protein; and optionally
(iii) further purifying and/or modifying and/or formulating the protein.

51. A method for treating a complement C3-mediated disease or disorder in a subject, comprising administering to a subject in need thereof the antigen binding protein or fragment thereof of claim 1, wherein optionally the antigen binding protein or fragment thereof is administered via topical, subconjunctival, intravitreal, retrobulbar, and/or intracameral administration; and/or wherein optionally the complement C3-mediated disease or disorder is selected from a group consisting age-related macular degeneration, geographic atrophy, neovascular glaucoma, diabetic retinopathy, retinopathy of prematurity, retrolental fibroplasia, autoimmune uveitis, chorioretinitis, retinitis, rheumatoid arthritis, psoriasis and atherosclerosis.

52-53. (canceled)

54. A method of inhibiting the activity of the complement Classical pathway (CP), Lectin pathway (LP), and Alternative pathway (AP), the method comprising contacting complement C3 with an antigen binding protein or fragment thereof which binds an epitope on complement C3; or inhibiting the activity of choroidal-localized complement C3, the method comprising intraocular administration of an antigen binding protein or fragment thereof which binds an epitope on complement C3.

55-69. (canceled)

70. A method of detecting one or both of C3 and C3b in a biological sample comprising the steps of:
(a) contacting the sample with at least one antigen binding protein or fragment thereof of claim 1;
(b) permitting formation of complexes between one or both of C3 and C3b and the antigen binding protein or fragment thereof in the sample; and
(c) detecting said antigen binding protein or fragment thereof;
wherein optionally the antigen binding protein or fragment thereof is capable of binding complement C3 and C3b; wherein optionally the antigen binding protein or fragment thereof is detected by a detectable signal; and/or wherein optionally the biological sample is a tissue sample, such as retinal tissue.

71-74. (canceled)


SEQUENCE LISTING (32 sequences)

SEQ ID NO: 1 - protein, 5 aa, synthetic construct
DYTMG

SEQ ID NO: 2 - protein, 17 aa, synthetic construct
AINWRGSSTYYADSVKG

SEQ ID NO: 3 - protein, 14 aa, synthetic construct
QVSPYVELTATAAY

SEQ ID NO: 4 - protein, 5 aa, synthetic construct
NWAMG

SEQ ID NO: 5 - protein, 17 aa, synthetic construct
AIRWSVGTTNYRDSVKG

SEQ ID NO: 6 - protein, 14 aa, synthetic construct
GTPFVLARINGYDY

SEQ ID NO: 7 - protein, 5 aa, synthetic construct
NYAMN

SEQ ID NO: 8 - protein, 16 aa, synthetic construct
IINTDGNTNYASWAKG

SEQ ID NO: 9 - protein, 11 aa, synthetic construct
AVGYHHHALDP

SEQ ID NO: 10 - protein, 12 aa, synthetic construct
TLSSAHKTYTID

SEQ ID NO: 11 - protein, 11 aa, synthetic construct
LKSDGSYTKGT

SEQ ID NO: 12 - protein, 9 aa, synthetic construct
GTDYGGGYV

SEQ ID NO: 13 - protein, 5 aa, synthetic construct
SYHMS

SEQ ID NO: 14 - protein, 16 aa, synthetic construct
IIYTDGNTDYANWAKG

SEQ ID NO: 15 - protein, 12 aa, synthetic construct
RGYADYGYTFNL

SEQ ID NO: 16 - protein, 11 aa, synthetic construct
TADTLSRNYAS

SEQ ID NO: 17 - protein, 7 aa, synthetic construct
RDTSRPS

SEQ ID NO: 18 - protein, 13 aa, synthetic construct
ATGDGSGSSYQFV

SEQ ID NO: 19 - protein, 5 aa, synthetic construct
RYWMN

SEQ ID NO: 20 - protein, 16 aa, synthetic construct
YITTNDKTYYANWAKG

SEQ ID NO: 21 - protein, 8 aa, synthetic construct
RSSGAYDI

SEQ ID NO: 22 - protein, 12 aa, synthetic construct
TLSSAHKTYYIE

SEQ ID NO: 23 - protein, 11 aa, synthetic construct
LKSDGTYTKGT

SEQ ID NO: 24 - protein, 9 aa, synthetic construct
GVTGGNVYV

SEQ ID NO: 25 - protein, 123 aa, synthetic construct
EVQLVESGGGLVQAGGSLRLSCAASGRTINDYTMGWFRQAPGKDREFVSAINWRGSSTYYADSVKGRFTISRDNAKKTIYLQMNLLKPEDTAVYYCARQVSPYVELTATAAYWGQGTQVTVSS

SEQ ID NO: 26 - protein, 123 aa, synthetic construct
EVQLVESGGGLVQAGGSLRLSCVASGHTFGNWAMGWFRQAPGKEREFVGAIRWSVGTTNYRDSVKGRFAISRDNARNTVYLQMNRLKPEDTAVYYCAAGTPFVLARINGYDYWGQGTQVTVSS

SEQ ID NO: 27 - protein, 117 aa, synthetic construct
QSVKESGGRLVTPGTPLTLTCTVSGFSLYNYAMNWVRQAPGKGLEWIGIINTDGNTNYASWAKGRFTISTTSSTTVDLKITSPTTEDTATYFCPRAVGYHHHALDPWGPGTLVTVSS

SEQ ID NO: 28 - protein, 112 aa, synthetic construct
ELVLTQSPSVSAALGASAKLTCTLSSAHKTYTIDWYQQQQGEAPRYLMQLKSDGSYTKGTGVPDRFSGSSSGADRYLIIPSVQADDEADYYCGTDYGGGYVFGGGTQLTVTG

SEQ ID NO: 29 - protein, 117 aa, synthetic construct
QSVKESEGRLVTPGTPLTLTCTASGFTIGSYHMSWVRQAPGKGLEWIGIIYTDGNTDYANWAKGRFTISKTSTTMDLKMTSLTAADTATYFCARRGYADYGYTFNLWGQGTLVTISS

SEQ ID NO: 30 - protein, 111 aa, synthetic construct
ELVLTQPASVQVNLGQTVSLTCTADTLSRNYASWYQQKPGQAPVLLIYRDTSRPSGVPDRFSGSSSGNTATLTISGAQAGDEADYYCATGDGSGSSYQFVFGGGTQLTVTG

SEQ ID NO: 31 - protein, 114 aa, synthetic construct
QSVKESGGRLVTPGTPLTLTCTVSGIDLSRYWMNWVRQAPGKGLEWIGYITTNDKTYYANWAKGRYTISKTSSTTVDLKMTSLTTEDTATYFCARRSSGAYDIWGPGTLVTISS

SEQ ID NO: 32 - protein, 112 aa, synthetic construct
QPVLTQSPSASATLGASAKLTCTLSSAHKTYYIEWYQQQQGEAPRYLMQLKSDGTYTKGTGVPDRFSGSSSGADRYLIISSVQAEDEADYICGVTGGNVYVFGGGTQLTVTG